(19) United States
(12) Patent Application Publication
(10) Pub. No.: US 2022/0370382 A1
(43) Pub. Date: Nov. 24, 2022

(54) METHODS AND COMPOSITIONS FOR CORRECTION OF AUTONOMIC DYSFUNCTIONS

(71) Applicant: Diana Driscoll, Keller, TX (US)

(72) Inventor: Diana Driscoll, Keller, TX (US)

(21) Appl. No.: 17/749,549

(22) Filed: May 20, 2022

Related U.S. Application Data
(60) Provisional application No. 63/191,753, filed on May 21, 2021.
(60) Provisional application No. 63/196,524, filed on Jun. 3, 2021.

(51) Int. Cl. A61K 31/14; A61K 45/06; A61P 9/00

(52) U.S. Cl. CPC A61K 31/14; A61K 45/06; A61P 9/00

(57) ABSTRACT

The present invention includes compositions and methods for treating certain conditions, the composition at least two active agents selected from: a choline compound; a cholinergic agonist; an acetylcholinesterase inhibitor (also referred to as a cholinesterase inhibitor); and a carnitine, in an amount effective to treat at least one of a connective tissue disorder, compression of or damage to any portion of a preganglionic or postganglionic vagus nerve, autonomic neuropathy, post-viral and post-infective autonomic dysfunction, post-traumatic autonomic dysfunction, physical trauma or mental/emotional trauma, post-traumatic stress disorder (PTSD), a genetic disorder of an acetylcholine cycle, inflammatory autonomic dysfunction, post-COVID (“Long-haulers”) or Post-Acute Sequelae SARS-CoV-2, or inflammatory Postural Orthostatic Tachycardia Syndrome (POTS).

### CROSS-REFERENCE TO RELATED APPLICATIONS

This application claims priority to U.S. Provisional Application Ser. No. 63/191,753, filed May 21, 2021, and U.S. Provisional Application Ser. No. 63/196,524, filed Jun. 3, 2021, the entire contents of each of which are incorporated herein by reference.

### TECHNICAL FIELD OF THE INVENTION

The present invention relates in general to the field of prevention and treatment of conditions associated with autonomic dysfunctions and their commonly found co-morbid conditions, signs or symptoms, and more particularly, to methods and composition for the prevention and treatment of the same.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with the diagnosis, treatment, and prevention of autonomic dysfunctions.

U.S. Pat. No. 10,238,673, issued to Driscoll and entitled, “Methods and compositions for treatment of dry eye and correction of organ dysfunctions,” teaches compositions and methods for treating certain conditions such as dry eye or dry mouth with a comprising a choline compound; a cholinesterase inhibitor; and Acetyl-L-Carnitine, wherein the composition is provided in an amount sufficient to treat dry eye or dry mouth.

U.S. Pat. No. 9,271,953, issued to Driscoll and entitled, “Methods for correction of organ dysfunction”, teaches compositions and methods for treating certain conditions, the composition comprising a choline compound; a cholinesterase inhibitor; and Acetyl-L-Carnitine, wherein the composition is used to treat at least one of autonomic dysfunctions or vascular diseases.

Another such method of treatment is taught in U.S. Pat. No. 7,599,736, issued to DiLorenzo, entitled “Method and apparatus for neuromodulation and physiologic modulation for the treatment of metabolic and neuropsychiatric disease.” Briefly, this inventor teaches an apparatus and method for physiological modulation, including neural and gastrointestinal modulation, for the purposes of treating several disorders, including obesity, depression, epilepsy, and diabetes. The method and apparatus includes a chronically implanted neural and neuromuscular modulator, used to modulate the afferent neurons of the sympathetic nervous system to induce satiety, including neuromuscular stimulation of the stomach to effect baseline and intermittent smooth muscle contraction to increase gastric intraluminal pressure, stimulation of sympathetic afferent fibers, including those in the sympathetic trunk, splanchnic nerves, and greater curvature of the stomach.

United States Patent Application No. 20110034376, filed by Lubbers, et al., is entitled, “Use of Lipid-Rich Nutrition for the Treatment of Post-Operative Ileus.” Briefly, the invention is directed to the use of a lipid-rich nutrition for the manufacture of a composition for the prevention and/or treatment of post-operative ileus. In peritoneal lavage fluid, the lipid fraction was said to inhibit IL-6 and TNF-alpha levels, wherein the lipid fraction prevents influx of neutrophils in the intestinal muscularis following intestinal manipulation.

United States Patent Application No. 20070093434, filed by Rossetti, et al., is entitled “Regulation of food intake and glucose production by modulation of long-chain fatty acyl-Co-A levels in the hypothalamus.” Briefly, the invention is directed to methods of reducing food intake and glucose production in a mammal, or restoring hepatic autoregulation are provided. The methods involve increasing long-chain fatty acyl-Co-A (LC-CoA) levels in the hypothalamus, or stimulating efferent fibers in the hepatic branch of the vagus nerve.

United States Patent Publication No. 20190298674, filed by During, entitled, “Use of (1S,3S)-3-AMINO-4-(DIFLUOROMETHYLIDENE) CYCLOPENTANE-1-CARBOXYLIC ACID AND (S)-3-AMINO-4-(DIFLUOROMETHYLENYL)CYCLOPENT-1-ENE-1-CARBOXYLIC ACID in the treatment of eye disorders”, is said to teach directed to methods of treating eye disorders with (S)-3-amino-4-(difluoromethylenyl)cyclopent-1-ene-1-carboxylic acid and therapeutic compositions that may be used to improve one or more symptoms of eye disorders.

### SUMMARY OF THE INVENTION

In one embodiment, the present invention includes a composition comprising: at least two active agents selected from: a choline compound; a cholinergic agonist; a cholinesterase inhibitor; and a carnitine, in an amount effective to treat at least one of a connective tissue disorder, compression of, damage to, or infection of, any portion of a preganglionic or postganglionic vagus nerve, autonomic dysfunction, autonomic neuropathy with partially functioning cholinergic receptors, post-viral and post-infective autonomic dysfunction, post-traumatic autonomic dysfunction, physical trauma or mental/emotional trauma, traumatic brain injury (TBI), post-traumatic stress disorder (PTSD), a genetic disorder of an acetylcholine production cycle, inflammatory autonomic dysfunction or inflammatory Postural Orthostatic Tachycardia Syndrome (POTS), chronic infectious and/or fatigue syndromes, Chronic Fatigue Syndrome, post-COVID (“Long-haulers”) or Post-Acute sequelae of COVID-19, Myalgic Encephalomyelitis, Chronic Lyme disease, or fibromyalgia. In one aspect, the connective tissue disorder is a disease associated with collagen, fibrillin, microfibrillin, elastin, or tenascin-X (genetic, acquired, or both). In another aspect, the autonomic dysfunction is due to or worsened by genetic disorders of inflammation (RCCX, CYP21A2, disorders of the TGF-beta cascade, MCP-1, TNX, SMAD), autoimmune disorders, abnormal hormones (androgens, estrogens or both), mast cell activation disorders, genetic disorders of tryptase, eosinophilic disorders (eosinophilic esophagitis, eosinophilic gastroenteritis, eosinophilia), adrenal disorders, congenital adrenal hyperplasia, Hyper IgE syndrome, low immune system (low IgG₁, IgG₂, IgG₃, IgG₄, or IgA), Idiopathic Intracranial Hypertension (IIH), post-infective inflammation (post-viral, post-SARS, post-COVID, or post-bacterial); vascular inflammation, vascular oxidation, hemochromatosis, hemolysis, reduced levels of nitric oxide, or impaired release of acetylcholine. In another aspect, the composition treats or prevents at least one of: autonomic dysfunction (dysautonomia, Postural Orthostatic Tachycardia Syndrome); multiple organ dysfunction (constipation, gastroparesis, idiopathic gastrointestinal dysmotility, low gastric acid production, ileocecal valve dysfunction (“ileus”), breathing difficulties, low gallbladder ejection fractions, biliary dyskinesia, acalculous gallbladder disease, Sphincter of Oddi dysfunction, low cholecystokinin (“CCK”) production, poor kidney function, non-alcoholic steatohepatitis (or “NASH”), or non-alcoholic fatty liver disease). In another aspect, the composition treats or prevents at least one of acquired disorders of connective tissue as seen in Ehlers-Danlos syndrome, Marfan's syndrome, Loeys-Dietz syndrome, Stickler syndrome, fibrillin disorders, elastin disorders, Joint Hypermobility Syndrome. In another aspect, the composition treats or prevents immune disorders, autoimmune disorders, autoinflammatory disorders, a vascular disease and a rheumatological disease. In another aspect, the composition treats or prevents dry eyes, xerostomia (dry mouth), vascular endothelial disorders, or poor nitric oxide production, chronic fungal infections, or hallucinations. In another aspect, the composition treats or prevents visual snow. In another aspect, the composition prevents or restores vascular endothelial health as determined by brachial artery ultrasound or spectral-domain optical coherence tomography, decreased thrombosis, improved vascular abnormalities, arrest of venous fibrosis as evidenced by ocular fundus photos or interventional vascular examination, elimination of orthostatic tachycardia, or reduction of livido reticularis. In another aspect, the acetylcholinesterase inhibitor (cholinesterase inhibitor) is selected from pyridostigmine, donepezil, tacrine, galantamine, and memantine, carbamates, physostigmine, neostigmine, ambenonium, demecarium, rivastigmine, phenanthrene derivatives, galantamine, caffeine, rosmarinic acid, alpha-pinene, piperidines, tacrine, edrophonium, huperzine A, ladostigil, ungeremine, lactucopicrin, or acotiamide. In another aspect, the cholinergic agonist is selected from cevimeline, carbamoylcholine, bethanechol, pilocarpine, varenicline, acetylcholine, arecoline, lobeline, GTS-21, or is provided as a nicotine patch. In another aspect, the carnitine is selected from L-carnitine, Acetyl L-carnitine, L-carnitine L-tartrate, or Propionyl-L-carnitine. In another aspect, the two active agents are selected from: the choline compound and the cholinergic agonist; the choline compound and the acetylcholinesterase inhibitor (or cholinesterase inhibitor); the choline compound and the carnitine; the cholinergic agonist and the acetylcholinesterase inhibitor; the cholinergic agonist and the carnitine; or the acetylcholinesterase inhibitor and the carnitine. In another aspect, the three active agents are selected from: the choline compound, the cholinesterase inhibitor and the cholinergic agonist; the choline compound, the acetylcholinesterase inhibitor and the carnitine; the choline, the cholinergic agonist, and the carnitine; or the acetylcholinesterase inhibitor, the cholinergic agonist and the carnitine. In another aspect, the four active agents selected from: the choline compound, the acetylcholinesterase inhibitor, the cholinergic agonist and the carnitine. In another aspect, the composition comprises the two or more active agents selected from: a choline compound at 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 3,750, or 4,000, or a range from 30 to 2,400, 40 to 2,300, 50 to 2,000, 60 to 1,500, 70 to 1,000, 80 to 750, 90 to 2,400, 200 to 2,300, 300 to 2,200, 400 to 2,100, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000, 1,600 to 2,100, 1,700 to 2,200, 1,800 to 2,300, or 1,900 to 2,400 mg of at least one choline; 0.001, 0.01, 0.1, 1, 2, 5, 10, 15, 20, 25, 30, 35, 40, 50 mg of the cholinesterase inhibitor selected from carbamates, physostigmine, neostigmine, pyridostigmine, ambenonium, demecarium, rivastigmine, phenanthrene derivatives, galantamine, caffeine (dose 1 mg-500 mg), rosmarinic acid, alpha-pinene, piperidines, donepezil, tacrine, edrophonium, huperzine A, ladostigil, ungeremine, lactucopicrin, memantine, or acotiamide; or 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 30 to 2,000, 40 to 2,300, 50 to 2,000, 60 to 1,500, 70 to 1,000, 80 to 750, 90 to 2,000, 200 to 2,000, 300 to 2,000, 400 to 2,000, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000 mg of the carnitine selected from L-carnitine, Acetyl L-carnitine, L-carnitine L-tartrate, or Propionyl-L-carnitine. In another aspect, the composition further comprises at least one of Thiamin, or Magnesium. In another aspect, the dose is 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 3,750, or 4,000, or a range from 30 to 2,400, 40 to 2,300, 50 to 2,000, 60 to 1,500, 70 to 1,000, 80 to 750, 90 to 2,400, 200 to 2,300, 300 to 2,200, 400 to 2,100, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000, 1,600 to 2,100, 1,700 to 2,200, 1,800 to 2,300, or 1,900 to 2,400 of the choline compound. In another aspect, the dose is 0.001, 0.01, 0.1, 1, 2, 5, 10, 15, 20, 25, 30, 35, 40, 50 mg of the cholinesterase inhibitor. In another aspect, the dose is 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 30 to 2,000, 40 to 2,300, 50 to 2,000, 60 to 1,500, 70 to 1,000, 80 to 750, 90 to 2,000, 200 to 2,000, 300 to 2,000, 400 to 2,000, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000 mg of the carnitine. In another aspect, the range per dose is 0.1, 0.5, 1, 5, 10, 20, 30, 40, 50, 60, 70, 80, 90, 100, 150, 200 mg Thiamin, and the range per dose is 0.1, 0.2, 0.3, 0.5, 0.75, 1.0, 5, 10, 15, 20, 25, 30, 40, 50, 60, 70, 80, 90, 100, 150, 200, 250, 300, 350, 400, 450, 500, 540, 600, 700, 750, 800 mg Magnesium. In another aspect, the composition is adapted to be administered prenatally, orally, intravenously, intraperitoneally, intranasally, intrapulmonary, subcutaneously, intracutaneously, or intramuscularly. In another aspect, the composition consists essentially of: two, three, or four active agents selected from: a choline compound; a cholinergic agonist; an acetylcholinesterase inhibitor; and a carnitine; and optionally including at least one of 0.1, 0.5, 1, 5, 10, 20, 30, 40, 50, 60, 70, 80, 90, 100, 150, 200 mg Thiamin, and the range per dose is 0.1, 0.2, 0.3, 0.5, 0.75, 1.0, 5, 10, 15, 20, 25, 30, 40, 50, 60, 70, 80, 90, 100, 150, 200, 250, 300, 350, 400, 450, 500, 540, 600, 700, 750, 800, mg Magnesium in an amount effective to treat at least one of a connective tissue disorder, compression of or damage to any portion of a preganglionic or postganglionic vagus nerve, autonomic neuropathy with partially functioning cholinergic receptors, post-viral (including post-SARS and post-COVID) and post-infective autonomic dysfunction, post-traumatic autonomic dysfunction, physical trauma or mental/emotional trauma, traumatic brain injury, post-traumatic stress disorder (PTSD), a genetic disorder of the acetylcholine manufacturing cycle, inflammatory autonomic dysfunction, or Inflammatory Postural Orthostatic Tachycardia Syndrome (POTS). In another aspect, the composition treats or prevents one or more signs or symptoms selected from: poor temperature control (poor thermoregulation), poor blood pressure control, large pupils, light sensitivity, poor ocular accommodation, early satiety, bloating, nausea, vomiting, difficulty swallowing, heartburn, delayed gastric emptying, loss of appetite, poor appetite regulation, diarrhea, constipation, gastric ilius, abdominal bloating, small intestine bacterial overgrowth (SIBO), small intestine fungal overgrowth (SIFO), dysbiosis, chronic candida, orthostatic hypotension, Postural Orthostatic Tachycardia Syndrome (POTS), exercise intolerance, dizziness, chronic fatigue, brain fog, diminished concentration, fainting, loss of short-term memory, loss of executive function, tachycardia, bradycardia, poor blood pressure regulation, orthostatic intolerance, anxiety, panic, panic attacks, flushing, environmental sensitivities, vascular endothelial damage, vascular leaking, dilated perivascular spaces, edema, clotting disorders, disseminated intravascular coagulation, paresthesia, visual disturbances, vision loss, visual snow, urinary retention, hallucinations (visual and auditory), disorientation, delirium, hyperactivity, restlessness, narrow angle glaucoma, muscle twitching, fasciculations, pseudoseizures, dysarthria, or poor coordination. In another aspect, the composition consists of: two, three, or four active agents selected from: a choline compound; a cholinergic agonist; an acetylcholinesterase inhibitor (cholinesterase inhibitor); and a carnitine; and optionally including at least one of 0.1-200 mg Thiamin or 1-800 mg Magnesium in an amount effective to treat at least one of a connective tissue disorder, compression of or damage to any portion of a preganglionic or postganglionic vagus nerve, autonomic neuropathy, autonomic neuropathy with partially functioning cholinergic receptors, postganglionic vagus nerve, or both), post-viral and post-infective autonomic dysfunction, post-traumatic autonomic dysfunction, physical trauma or mental/emotional trauma, post-traumatic stress disorder (PTSD), a genetic disorder of an acetylcholine cycle, inflammatory autonomic dysfunction, or inflammatory Postural Orthostatic Tachycardia Syndrome (POTS).

In another embodiment, the present invention includes a method of treating a disease or condition in a subject, comprising administering to a subject in need thereof an effective amount of a composition comprising at least two active agents selected from: a choline compound; a cholinergic agonist; an acetylcholinesterase inhibitor (cholinesterase inhibitor); and a carnitine, in an amount effective to treat at least one of a connective tissue disorder, compression of, damage to, or infection of, any portion of a preganglionic or postganglionic vagus nerve, autonomic dysfunction, autonomic neuropathy with partially functioning cholinergic receptors, postganglionic vagus nerve, post-viral and post-infective autonomic dysfunction, post-traumatic autonomic dysfunction, physical trauma or mental/emotional trauma, traumatic brain injury (TBI), post-traumatic stress disorder (PTSD), a genetic disorder of an acetylcholine production cycle, inflammatory autonomic dysfunction or inflammatory Postural Orthostatic Tachycardia Syndrome (POTS), chronic infectious and/or fatigue syndromes, Chronic Fatigue Syndrome, post-COVID (“Long-haulers”) or Post-Acute sequelae of COVID-19, Myalgic Encephalomylitis, Chronic Lyme disease, or fibromyalgia. In one aspect, the connective tissue disorder is a disease associated with collagen, fibrillin, microfibrillin, elastin, or tenascin-X (genetic, acquired, or both). In another aspect, the autonomic dysfunction is due to or worsened by genetic disorders of inflammation (RCCX, CYP21A2, disorders of the TGF-beta cascade, MCP-1, TNX, SMAD), autoimmune disorders, abnormal hormones (androgens, estrogens or both), mast cell activation disorders, genetic disorders of tryptase, eosinophilic disorders (eosinophilic esophagitis, eosinophilic gastroenteritis, eosinophilia), adrenal disorders, congenital adrenal hyperplasia, Hyper IgE syndrome, low immune system (low IgG₁, IgG₂, IgG₃, IgG₄, or IgA), Idiopathic Intracranial Hypertension (IIH), post-infective inflammation (post-viral, post-SARS, post-COVID, or post-bacterial); vascular inflammation, vascular oxidation, hemochromatosis, hemolysis, reduced levels of nitric oxide, or impaired release of acetylcholine. In another aspect, the composition treats or prevents at least one of: autonomic dysfunction (dysautonomia, Postural Orthostatic Tachycardia Syndrome); multiple organ dysfunction (constipation, gastroparesis, idiopathic gastrointestinal dysmotility, low gastric acid production, ileocecal valve dysfunction (“ileus”), breathing difficulties, low gallbladder ejection fractions, biliary dyskinesia, acalculous gallbladder disease, Sphincter of Oddi dysfunction, low cholecystokinin (“CCK”) production, poor kidney function, non-alcoholic steatohepatitis (or “NASH”), or non-alcoholic fatty liver disease). In another aspect, the composition treats or prevents at least one of acquired disorders of connective tissue as seen in Ehlers-Danlos syndrome, Marfan's syndrome, Loeys-Dietz syndrome, Stickler syndrome, fibrillin disorders, elastin disorders, Joint Hypermobility Syndrome. In another aspect, the composition treats or prevents immune disorders, autoimmune disorders, autoinflammatory disorders, a vascular disease and a rheumatological disease. In another aspect, the composition treats or prevents dry eyes, xerostomia (dry mouth), vascular endothelial disorders, or poor nitric oxide production, chronic fungal infections, or hallucinations. In another aspect, the composition treats or prevents visual snow. In another aspect, the composition prevents or restores vascular endothelial health as determined by brachial artery ultrasound or spectral-domain optical coherence tomography, decreased thrombosis, improved vascular abnormalities, arrest of venous fibrosis as evidenced by ocular fundus photos or interventional vascular examination, elimination of orthostatic tachycardia, or reduction of livido reticularis. In another aspect, the acetylcholinesterase inhibitor (cholinesterase inhibitor) is selected from pyridostigmine, donepezil, tacrine, galantamine, and memantine, carbamates, physostigmine, neostigmine, ambenonium, demecarium, rivastigmine, phenanthrene derivatives, galantamine, caffeine, rosmarinic acid, alpha-pinene, piperidines, tacrine, edrophonium, huperzine A, ladostigil, ungeremine, lactucopicrin, or acotiamide. In another aspect, the cholinergic agonist is selected from cevimeline, carbamoylcholine, bethanechol, pilocarpine, varenicline, acetylcholine, arecoline, lobeline, GTS-21, or is provided as a nicotine patch. In another aspect, the carnitine is selected from L-carnitine, Acetyl L-carnitine, L-carnitine L-tartrate, or Propionyl-L-carnitine. In another aspect, the two active agents are selected from: the choline compound and the cholinergic agonist; the choline compound and the acetylcholinesterase inhibitor (or cholinesterase inhibitor); the choline compound and the carnitine; the cholinergic agonist and the acetylcholinesterase inhibitor; the cholinergic agonist and the carnitine; or the acetylcholinesterase inhibitor and the carnitine. In another aspect, the three active agents are selected from: the choline compound, the cholinesterase inhibitor and the cholinergic agonist; the choline compound, the acetylcholinesterase inhibitor and the carnitine; the choline, the cholinergic agonist, and the carnitine; or the acetylcholinesterase inhibitor, the cholinergic agonist and the carnitine. In another aspect, the four active agents selected from: the choline compound, the acetylcholinesterase inhibitor, the cholinergic agonist and the carnitine. In another aspect, the composition comprises the two or more active agents selected from: a choline compound at 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 3,750, or 4,000, or a range from 30 to 2,400, 40 to 2,300, 50 to 2,000, 60 to 1,500, 70 to 1,000, 80 to 750, 90 to 2,400, 200 to 2,300, 300 to 2,200, 400 to 2,100, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000, 1,600 to 2,100, 1,700 to 2,200, 1,800 to 2,300, or 1,900 to 2,400 mg of at least one choline; 0.001, 0.01, 0.1, 1, 2, 5, 10, 15, 20, 25, 30, 35, 40, 50 mg of the cholinesterase inhibitor selected from carbamates, physostigmine, neostigmine, pyridostigmine, ambenonium, demecarium, rivastigmine, phenanthrene derivatives, galantamine, caffeine (dose 1 mg-500 mg), rosmarinic acid, alpha-pinene, piperidines, donepezil, tacrine, edrophonium, huperzine A, ladostigil, ungeremine, lactucopicrin, memantine, or acotiamide; or 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 3,500, 3,750, or 4,000, or a range from 30 to 2,000, 40 to 2,300, 50 to 2,000, 60 to 1,500, 70 to 1,000, 80 to 750, 90 to 2,000, 200 to 2,000, 300 to 2,000, 400 to 2,000, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000 mg of the carnitine selected from L-carnitine, Acetyl L-carnitine, L-carnitine L-tartrate, or Propionyl-L-carnitine. In another aspect, the composition further comprises at least one of Thiamin, or Magnesium. In another aspect, the dose is 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 3,750, or 4,000, or a range from 30 to 2,400, 40 to 2,300, 50 to 2,000, 60 to 1,500, 70 to 1,000, 80 to 750, 90 to 2,400, 200 to 2,300, 300 to 2,200, 400 to 2,100, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000, 1,600 to 2,100, 1,700 to 2,200, 1,800 to 2,300, or 1,900 to 2,400 of the choline compound. In another aspect, the dose is 0.001, 0.01, 0.1, 1, 2, 5, 10, 15, 20, 25, 30, 35, 40, 50 mg of the cholinesterase inhibitor. In another aspect, the dose is 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 30 to 2,000, 40 to 2,300, 50 to 2,000, 60 to 1,500, 70 to 1,000, 80 to 750, 90 to 2,000, 200 to 2,000, 300 to 2,000, 400 to 2,000, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000 mg of the carnitine. In another aspect, the range per dose is 0.1, 0.5, 1, 5, 10, 20, 30, 40, 50, 60, 70, 80, 90, 100, 150, 200 mg Thiamin, and the range per dose is 0.1, 0.2, 0.3, 0.5, 0.75, 1.0, 5, 10, 15, 20, 25, 30, 40, 50, 60, 70, 80, 90, 100, 150, 200, 250, 300, 350, 400, 450, 500, 540, 600, 700, 750, 800, mg Magnesium. In another aspect, the composition is adapted to be administered prenatally, orally, intravenously, intraperitoneally, intranasally, intrapulmonary, subcutaneously, intracutaneously, or intramuscularly. In another aspect, the composition consists essentially of: two, three, or four active agents selected from: a choline compound; a cholinergic agonist; an acetylcholinesterase inhibitor; and a carnitine; and optionally including at least one of 0.1, 0.5, 1, 5, 10, 20, 30, 40, 50, 60, 70, 80, 90, 100, 150, 200 mg Thiamin, and the range per dose is 0.1, 0.2, 0.3, 0.5, 0.75, 1.0, 5, 10, 15, 20, 25, 30, 40, 50, 60, 70, 80, 90, 100, 150, 200, 250, 300, 350, 400, 450, 500, 540, 600, 700, 750, 800, mg Magnesium in an amount effective to treat at least one of a connective tissue disorder, compression of or damage to any portion of a preganglionic or postganglionic vagus nerve, autonomic neuropathy with partially functioning cholinergic receptors, post-viral (including post-SARS and post-COVID) and post-infective autonomic dysfunction, post-traumatic autonomic dysfunction, physical trauma or mental/emotional trauma, traumatic brain injury, post-traumatic stress disorder (PTSD), a genetic disorder of the acetylcholine manufacturing cycle, inflammatory autonomic dysfunction, or Inflammatory Postural Orthostatic Tachycardia Syndrome (POTS). In another aspect, the composition treats or prevents one or more signs or symptoms selected from: poor temperature control (poor thermoregulation), poor blood pressure control, large pupils, light sensitivity, poor ocular accommodation, early satiety, bloating, nausea, vomiting, difficulty swallowing, heartburn, delayed gastric emptying, loss of appetite, poor appetite regulation, diarrhea, constipation, gastric ilius, abdominal bloating, small intestine bacterial overgrowth (SIBO), small intestine fungal overgrowth (SIFO), dysbiosis, chronic candida, orthostatic hypotension, Postural Orthostatic Tachycardia Syndrome (POTS), exercise intolerance, dizziness, chronic fatigue, brain fog, diminished concentration, fainting, loss of short-term memory, loss of executive function, tachycardia, bradycardia, poor blood pressure regulation, orthostatic intolerance, anxiety, panic, panic attacks, flushing, environmental sensitivities, vascular endothelial damage, vascular leaking, dilated perivascular spaces, edema, clotting disorders, disseminated intravascular coagulation, paresthesia, visual disturbances, vision loss, visual snow, urinary retention, hallucinations (visual and auditory), disorientation, delirium, hyperactivity, restlessness, narrow angle glaucoma, muscle twitching, fasciculations, pseudoseizures, dysarthria, or poor coordination. In another aspect, the composition consists of: two, three, or four active agents selected from: a choline compound; a cholinergic agonist; an acetylcholinesterase inhibitor (cholinesterase inhibitor); and a carnitine; and optionally including at least one of 0.1-200 mg Thiamin or 1-800 mg Magnesium in an amount effective to treat at least one of a connective tissue disorder, compression of or damage to any portion of a preganglionic or postganglionic vagus nerve, autonomic neuropathy, autonomic neuropathy with partially functioning cholinergic receptors, postganglionic vagus nerve, or both), post-viral and post-infective autonomic dysfunction, post-traumatic autonomic dysfunction, physical trauma or mental/emotional trauma, post-traumatic stress disorder (PTSD), a genetic disorder of an acetylcholine cycle, inflammatory autonomic dysfunction, or inflammatory Postural Orthostatic Tachycardia Syndrome (POTS).

In another embodiment, the present invention includes a method for diagnosis and treatment of a compression of or damage to any portion of a preganglionic or postganglionic vagus nerve, or a post-viral and post-infective autonomic dysfunction, in a subject comprising: identifying a subject that has one or more symptoms associated with compression of or damage to any portion of a preganglionic or postganglionic vagus nerve, or a post-viral and post-infective autonomic dysfunction, selected from at least one of: constipation, gastroparesis, idiopathic gastrointestinal dysmotility, ileocecal valve dysfunction (“ileus”), low stomach acid production, low gallbladder ejection fractions, pancreatic enzyme deficiency, tachycardia, or low acetylcholine release is suspected based on symptoms of anticholinergic syndrome; applying to the subject a nicotine patch; determining if the subject has a reduction in the one or more symptoms, and if the subject has a reduction of the one or more symptoms, then providing the subject with a composition of claim 1. In one aspect, the symptoms are determined by at least one of: a bowel movement journal reflects constipation (fewer than one bowel movement per day); Small bowel manometry reflects low motility; Imaging with fluoroscopy reveals ileocecal valve dysfunction (ileus); Heidelberg testing reveals low stomach acid production; HIDA scan reveals low gallbladder ejection fraction; stool quality or color, steatorrhea—oil at the top of the toilet water after a bowel movement, reveals pancreatic enzyme deficiency; or Tachycardia at rest. In another aspect, the nicotine patch stimulates post-ganglionic nicotinic acetylcholine receptors or the nicotinic acetylcholine receptors on the organs that results in correction of at least one of: constipation, gastroparesis, idiopathic gastrointestinal dysmotility, ileocecal valve dysfunction (“ileus”), low stomach acid production, low gallbladder ejection fractions, pancreatic enzyme deficiency, or tachycardia. In another aspect, the nicotine patch is placed on an abdomen or trunk. In another aspect, the method further comprises checking the effect of the abnormal testing for normalization after 30 minutes, 2, 3, 4, 5, 6, 7, 8, 9, 10, 11, 12, 13, 14, 15, 16, 17, 18, 19, 20, 21, 22, 23, or 24 hours. In another aspect, a response to the nicotine patch is indicative that a post-ganglionic receptor is viable, wherein a subject is provided a composition comprising at least two active agents selected from: a choline compound; a cholinergic agonist; a cholinesterase inhibitor; and a carnitine.

### BRIEF DESCRIPTION OF THE DRAWINGS

For a more complete understanding of the features and advantages of the present invention, reference is now made to the detailed description of the invention along with the accompanying figures and in which:

FIG. 1 shows blue arrows that point to premature plaque formation in arterioles with narrowed vascular lumen.

FIG. 2 shows blue arrows that point to premature plaque formation in arterioles with narrowed vascular lumen.

FIGS. 3A and 3B show black arrow that point to venous fibrosis evidenced by irregular caliber.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a” “an” and “the” are not intended to refer to only a singular entity, but include the general class of which a specific example may be used for illustration. The terminology herein is used to describe specific embodiments of the invention, but their usage does not delimit the invention, except as outlined in the claims. N/A—not applicable.

The present inventor has recognized a need to treat and prevent autonomic dysfunction (dysautonomia, postural orthostatic tachycardia syndrome) and its associated conditions beyond the conditions treated by the compound described by Driscoll, U.S. Pat. No. 9,271,953. Conditions treatable are not limited to collagen disorders and instead include connective tissue disorders such as genetic and/or acquired disorders of connective tissue (which may include most forms of Ehlers-Danlos syndrome, Marfan's syndrome, Loeys-Dietz syndrome, Stickler syndrome, fibrillary disorders, elastin disorders, Joint Hypermobility Syndrome disorders of fibrillin and microfibrillin, Tenascin-X, and elastin), acquired disorders of connective tissue to include inflammatory states, mast cell activation syndrome (MCAS), chronic infectious and/or fatigue syndromes (which may include Chronic Fatigue Syndrome, post-COVID (“Long-haulers”) or Post-Acute sequelae of COVID-19, Myalgic Encephalomylitis, Chronic Lyme disease, fibromyalgia) adrenal disease, congenital adrenal hyperplasia, eosinophilic activation disorders (including eosinophilic esophagitis and eosinophilic gastroenteritis, eosinophilic disorders secondary to Transforming Growth Factor-beta, autoinflammatory conditions, autoimmune disorders including early Sjogren's syndrome, a vascular endothelial disease (including atherosclerosis, vascular endothelial damage, disseminated intravascular coagulation, leaky vessels, dilated perivascular spaces) and a rheumatological disease.

The present inventor also recognized that a need exists for the treatment of the root causes of Postural Orthostatic Tachycardia Syndrome (“POTS”), post-viral and post-infective autonomic dysfunction, chronic dry eyes, visual snow, and idiopathic dysautonomia (disorders of the autonomic nervous system). These chronic conditions possess some commonalities, but treatment based on the underlying medical origination of illness are lacking. Associated conditions include traumatic brain injury (TBI), Post Traumatic Stress Disorder (PTSD)), post-infectious Postural Orthostatic Tachycardia Syndrome, inflammatory autonomic dysfunction and Inflammatory Postural Orthostatic Tachycardia Syndrome, genetic disorders of RCCX, CYP21A2, disorders of the TGF-beta cascade, TNX, SMAD), autoimmune disorders, abnormal hormones (androgens and estrogens), genetic disorders of tryptase (including hereditary alpha tryptasemia), Hyper IgE Syndrome, low immune system (low IgG, low IgA), Idiopathic Intracranial Hypertension (IIH), vascular oxidation (including that due to hemochromatosis and hemolysis), low nitric oxide, impaired release of acetylcholine, genetic disorders of the acetylcholine manufacturing cycle, compression of or damage to any portion of the preganglionic or postganglionic vagus nerve, autonomic neuropathy (with partially functioning cholinergic receptors and/or postganglionic vagus nerve). These conditions result in a vast array of symptoms and signs involving organ dysfunction, low nitric oxide production, vascular endothelial damage, clotting disorders, visual snow dry eyes, xerostomia (dry mouth). The present invention is uniquely designed to simultaneously correct or control the underlying causes and associated conditions sufficiently to arrest the progression of illness resulting in this vast array of symptoms and signs.

Specifically, it has been found that the present invention provides almost immediate treatment for the symptoms and medical conditions associated with the various medical diseases without the adverse side effects common to the use of the components of the composition at different doses. It has also been found that the composition functions in a manner superior to the individual components. Surprisingly, it was also found that the listed doses avoided adverse side effects from the use of the same components alone and in different amounts. The only exception being the transdermal, vaginal suppository, or anal suppository form of nicotine, which can be used as a diagnostic tool to diagnose the origin of poor vagus nerve function. These forms of nicotine can be used to discern receptor damage from nerve damage or neurotransmitter dysfunction. These forms of nicotine can be used to verify that the organ is capable of responding, to test the cholinergic receptors or postganglionic vagus nerve, or can be used for short periods of time to assist with said organ function (ileocecal valve, gastroparesis/constipation, gallbladder, pancreas, gastric acid secretion).

This patent includes compositions, methods of treatment and methods for preventing, diagnosing and treating autonomic dysfunction and its associated conditions and co-morbid presentations as is seen in disorders of the autonomic nervous system. Such disorders result in a vast array of symptoms and signs including constipation, gastroparesis, idiopathic gastrointestinal dysmotility, low gastric acid production, ileocecal valve dysfunction, gastric ileus, breathing difficulties, low gallbladder ejection fractions, biliary dyskinesia, acalculous gallbladder disease, Sphincter of Oddi dysfunction, pyloric valve dysfunction, low cholecystokinin (“CCK”) production, non-alcoholic steatohepatitis (or “NASH”), non-alcoholic fatty liver disease, in addition to poor temperature control (poor thermoregulation), poor blood pressure control, large pupils, light sensitivity, poor ocular accommodation, early satiety, bloating, nausea, vomiting, difficulty swallowing, heartburn, delayed gastric emptying, loss of appetite, poor appetite regulation, diarrhea, constipation, abdominal bloating, SIBO (small intestine bacterial overgrowth), SIFO (small intestine fungal overgrowth), dysbiosis, chronic candida, orthostatic hypotension, exercise intolerance, dizziness, chronic fatigue, brain fog, diminished concentration, fainting, loss of short-term memory, loss of executive function, tachycardia, bradycardia, poor blood pressure regulation, orthostatic intolerance, anxiety, panic, panic attacks, flushing, environmental sensitivities, vascular endothelial damage, vascular leaking, edema, clotting disorders, disseminated intravascular coagulation, paresthesia, visual disturbances, vision loss, visual snow, urinary retention, hallucinations (visual and auditory), disorientation, delirium, hyperactivity, restlessness, narrow angle glaucoma, muscle twitching, fasciculations, pseudoseizures, dysarthria, and poor coordination. These associated conditions are not limited to the autonomic nervous system and can affect the central nervous system and the peripheral nervous system. This composition corrects this autonomic dysfunction, in addition to the effects on the peripheral and central nervous system. Organs recover their function (unless they are anatomically damaged beyond the ability to even partially function, or unless the receptors on the organs are destroyed and unable to regenerate).

Although a variety of genetic, vascular and neurological processes contribute to the autonomic dysfunction, this composition is made to correct (and/or work around) the majority of defects present, most of which result in symptoms and signs of low parasympathetic nervous system function including those of anticholinergic syndrome, in addition to the numerous associated conditions listed above.

Poor parasympathetic nervous system functioning results in the environment needed for gastrointestinal inflammation that can reduce nutrient absorption. Proper digestion depends upon the parasympathetic nervous system and includes gastric acid secretion, movement of food, chyme, and stool through the gastrointestinal tract, gallbladder bile release, opening of the ileocecal valve, pancreatic digestive enzyme release, and opening of the Sphincter of Oddi. When dysautonomia results in a poorly functioning parasympathetic nervous system, mast cell activation of the mucocutaneous surfaces, Small Intestine Bacterial Overgrowth (SIBO), Small Intestine Fungal Overgrowth (SIFO), eosinophilic gastroenteritis, colitis, and other forms of gastrointestinal inflammation result. Ironically, and not obvious to those in the art, it was found by the present inventor that bowel disorders secondary to inflammatory cytokines or cells interfering with the release of acetylcholine by the parasympathetic nervous system including the vagus nerve, or damage to the preganglionic or postganglionic parasympathetic nerves eventually leads to organ dysfunction, chronic dry eyes, and visual snow that is reversible by the invention. Browning K N, Travagli R A. Central nervous system control of gastrointestinal motility and secretion and modulation of gastrointestinal functions. Compr Physiol. 2014 October; 4(4):1339-68. doi: 10.1002/cphy.c130055. PMID: 25428846: PMCID: PMC4858318.

Dysfunction of the autonomic nervous system is suspected or diagnosed in patients with abnormal heart rate variability tests, tilt table tests, sweat tests (sudomotor testing), and/or thermoregulatory testing or chronic dry eyes, often with enlarged pupils and light sensitivity. Vagus nerve dysfunction can be caused by vagus nerve compression by enlarged internal jugular veins. Driscoll D. The Driscoll Theory: Discovering the Cause of POTS in Ehlers-Danlos Syndrome. Warnick Publishing, 2012. Print Vagus nerve malfunction or desensitization and can be induced by injury (such as trauma, surgery, “nerve stretch” injuries, whiplash, vagotomy, spinal cord injury, compression by bones or abnormal calcium deposition or growths, abdominal, thoracic and/or heart surgery, “lap-band”, gastric sleeve or other surgeries of the stomach), opiate medications. Parasympathetic nerves can be rendered ineffective either by injury or by surrounding inflammatory cells and/or cytokines inhibiting the release of acetylcholine (the neurotransmitter utilized by these parasympathetic nerves). When the effector organ is anatomically and structurally capable of functioning, this composition is uniquely capable of stimulating the (undamaged) post-ganglionic nicotinic acetylcholine receptor or the organ's receptor, triggering normal organ response.

Peltier A C, Garland E, Raj S R, Sato K, Black B, Song Y, Wang L, Biaggioni I, Diedrich A, Robertson D. Distal sudomotor findings in postural tachycardia syndrome. Clin Auton Res. 2010 April; 20(2):93-9. doi: 10.1007/s10286-009-0045-y. Epub 2009 Dec. 25. PMID: 20035362; PMCID: PMC3089763.

When the preganglionic parasympathetic nerve is damaged or otherwise non-functional, this composition and method takes advantage of the often viable postganglionic vagus nerve in these conditions (or to the nicotinic acetylcholine receptors themselves), and mimics the neuroreceptor release by a healthy nerve, allowing for triggering of the post-ganglionic vagus nerve or the organ's receptors, and resulting in organ function.

It is understood by those familiar in the art that some inflammatory cytokines and inflammatory cells can block the release of acetylcholine by the pre- or post-ganglionic vagus nerve (rendering the nerve function deficient). Ironically and importantly, the vagus nerve is the anti-inflammatory nerve of the body. This cycle of inflammation leading to vagus nerve dysfunction, ultimately resulting in more inflammation, must be controlled. This invention restores function of the vagus nerve despite the reduced release of acetylcholine, and this ultimately controls the release of inflammatory cytokines. Uniquely, this invention does not require a functioning vagus nerve to be effective. In the case of vagus nerve damage (pre- and post-ganglionic damage), the invention stimulates the receptor directly on the organ. The inventor has found that in cases where lymphocytes and/or inflammatory cytokines are blocking the release of acetylcholine, nerve stimulation is not effective—no organ response is measured. As found by Zoukhn and Kublin, when lymphocytes block the release of acetylcholine at the lacrimal nerve in early Sjogren's, stimulation of the lacrimal nerve is insufficient to allow the release of the neuroreceptor. Zoukhri D, Kublin C L. Impaired neurotransmitter release from lacrimal and salivary gland nerves of a murine model of Sjogren's syndrome. Investigative Ophthalmology & Visual Science. 2001 April; 42(5):925-932. In a similar fashion, in the case of vagus nerve damage (structural damage, such as vagotomy), stimulation of the nerve is also not effective. The invention works around all of these situations uniquely, by stimulating the receptors on the effector organ—and this is accomplished with an oral blend.

References Han B, Li X, Hao J. The cholinergic anti-inflammatory pathway: An innovative treatment strategy for neurological diseases. Neurosci Biobehav Rev. 2017 June; 77:358-368. doi: 10.1016/j.neubiorev.2017.04.002. Epub 2017 Apr. 6. PMID: 28392244. Bonaz B, Sinniger V, Pellissier S. Anti-inflammatory properties of the vagus nerve: potential therapeutic implications of vagus nerve stimulation. J Physiol. 2016 Oct. 15; 594(20):5781-5790. doi: 10.1113/JP271539. Epub 2016 May 1. PMID: 27059884; PMCID: PMC5063949.

Some inflammatory cytokines and inflammatory cells can damage the vascular endothelium. The number of methods to diagnose vascular endothelial damage is limited and includes brachial artery ultrasound and the cold processor test. These are limited by user technique and patient discomfort. Importantly, these methods do not diagnose venous endothelial damage—they only measure the arterial endothelial health. The inventor has found that these tests are less sensitive when the patient does not have advanced atherosclerosis. Instead, the inventor utilized ocular fundus photos and found that the majority of patients tested with autonomic dysfunction displayed narrow arteriole lumen with visible atherosclerotic changes in the ocular fundus (noticed even in children with dysautonomia), reflecting vascular inflammation. Importantly, ocular fundus photos also reveal abnormal venous caliber, reflecting venous fibrosis and poor endothelial health of the veins and venules. These abnormalities are apparent with both traditional fundus photography and with spectral-domain optical coherence tomography. The present invention aborts vascular damage due to poorly controlled inflammation secondary to low parasympathetic and vagus nerve function. It accomplishes this by activating the vagus nerve's function (the anti-inflammatory nerve of the body) as well as by promoting acetylcholine levels which are necessary for vascular health (acetylcholine stimulates eNOS to promote the conversion of L-arginine to L-citrulline and nitric oxide). Uniquely, this invention promotes acetylcholine, despite any damage to autonomic nervous system, ultimately repairing vascular endothelial damage, and often dramatically improving orthostatic tolerance in those with autononic dysfunction.

Reference: Zhu T P, Tong Yi, Zhan H J, Ma J. Update on retinal vessel structure measurement with spectral-domain optical coherence tomography. Microvasc Res. 2014 September:95:7-14. doi: 10 1016/j.mvr2014.06.007. Epub 2014 Jun. 27. PMID: 24976361. Reference for the statement about acetylcholine stimulating eNOS: Schwartz, B., Economides, C., Mayeda, G. et a. The endothelial cell in health and disease: its function, dysfunction, measurement and therapy. Int J Impol Res 22, 77-90 (2010). https://doi.org/10.1038/ijir.2009.59.

Certain ingredients of the compound (a choline compound, an acetylcholinesterase inhibitor and Acetyl-L-Carnitine) have been used to reduce inflammation, to assist those suffering from metabolic insufficiencies, and for healthy memory and mental function, and numerous studies have been published involving cognition, dementia, age-related memory loss and neurodegeneration with these ingredients. (U.S. Pat. No. 6,537,969 B1, Patent No. US 2008/0213401). The present inventor has previously found that the combination of these 3 ingredients, when blended appropriately and in the proper proportions could stimulate the postganglionic portion of the vagus nerve, resulting in a bowel movement (and allowing normal organ function). It is not intuitive or obvious that any two of these ingredients can also be effective and that any two of these ingredients can restore parasympathetic nerve function even when the postganglionic nerve is not viable for any reason, by directly stimulating the effector organs themselves. Any one ingredient, however, was found to be insufficient to restore vagus nerve function.

The present invention can also be used to treat “visual snow”, a visual phenomenon that presents much like static on a television and is distinct from common ocular vitreous floaters or after-images. It was found that 27% of 192 patients polled with autonomic dysfunction (POTS—Postural Orthostatic Tachycardia Syndrome) reported experiencing true visual snow. Resolution of visual snow can occur with use of this compound. Interestingly, the majority of patients presenting with visual snow and autonomic dysfunction also presented with Idiopathic Intracranial Hypertension and for complete resolution of visual snow, reduction of cerebrospinal fluid pressure (via acetazolamide, furosemide, or cerebrospinal fluid withdraw) was often necessary, in addition to the invention. Cerebrospinal fluid pressure reduction was insufficient, however, for resolution of visual snow without the use of the compound.

Not intending to be bound by theory, and in no way a limitation of the present invention, the invention (excluding nicotine) can be used to stimulate production of saliva by the salivary glands by stimulating the receptors on the salivary glands. This composition can be used to treat xerostomia (dry mouth) when the salivary glands and acinar cells are viable. Chronic dry eyes and dry mouth is common in dysautonomia, Postural Orthostatic Tachycardia Syndrome, and in connective tissue disorders.

When the vagus nerve is underperforming for any reason, nutrient malabsorption can result. When deficiencies of magnesium and/or Thiamin occur, the production of acetylcholine can be affected, further reducing parasympathetic nervous system function. Magnesium deficiency is believed to be a possible cause of thiamin deficiency. Thiamin is essential for acetylcholine production. Uniquely, the cycle of poor parasympathetic nervous system function leading to malabsorption, contributing to low acetylcholine production, further reducing parasympathetic nervous system function can be aborted with the use of this compound (or nicotine in the case of vagus nerve dysfunction). Ruenwongsa P, Pattanavibag S. Impairment of acetylcholine synthesis in thiamine deficient rats developed by prolonged tea consumption. Life Sci. 1984 Jan. 23; 34(4):365-70. doi: 10.1016/0024-3205(84)90625-8. PMID: 6694525.

Driscoll found the use of nicotine can diagnose pre-ganglionic vagus nerve dysfunction. By contrast, it is found herein that nicotine can be used as a diagnostic tool to diagnose the origin of poor vagus nerve function (which is more specific, unique, and not intuitive). Nicotine (transdermal, suppository, or) can be used to discern receptor damage from either direct nerve damage or neurotransmitter dysfunction. These forms of nicotine can be used to verify that the organ is capable of responding, to test the cholinergic receptors or postganglionic nerve, or can be used for short periods of time to assist with said organ function (ileocecal valve, gastroparesis/constipation, gallbladder, pancreas, gastric acid secretion). No part of the vagus nerve is required for the use of nicotine to be effective because the invention can effectively stimulate the receptor on the organ itself. Therefore, a unique method for diagnosing vagus nerve dysfunction (when there is little or no damage to nicotinic acetylcholine receptors on the effector organs, involves stimulation of the nicotinic acetylcholine receptors via the use of transdermal nicotine (1-21 mg nicotine) applied to the abdomen. If the nicotinic acetylcholine receptor on the organ is viable, nicotine (acting as an agonist for acetylcholine at the nicotinic acetylcholinergic receptor) will reverse symptoms and signs of vagus nerve dysfunction including chronic constipation, gastroparesis, idiopathic gastrointestinal dysmotility, ileocecal valve dysfunction (“ileus”), and Sphincter of Oddi dysfunction (when the sphincter is not damaged or blocked) usually within hours. Such stimulation via nicotine can be used to verify that this aspect of autonomic dysfunction is secondary to either vagus nerve damage or neurotransmitter dysfunction—but it is not due to an ineffective receptor (the receptor is viable), and now that this is diagnosed, the compound can be substituted for nicotine. Oral nicotine is not effective in stimulating these receptors sufficiently to result in normalized organ function. It is not intuitive, obvious or previously discovered that two ingredients of the oral formulation consisting of a choline compound, a cholinesterase compound and a carnitine will stimulate the nicotinic-acetylcholine receptors directly on the effector organs, resulting in organ response.

Nicotine as a treatment of parasympathetic and vagus nerve dysfunction cannot be used on a chronic basis because of side effects (the enhancement of histamine expression of cyclooxygenase-2 in endothelial cells resulting in dermatographia, hives, itching, and welting). Dermatographia has been found by the inventor to affect 82% of patients with autonomic dysfunction and/or connective tissue disorders. Instead, nicotine can be used as a (short-term) diagnostic tool (to test for viability of nicotinic receptors) or a short-term treatment only for pre- or post-ganglionic vagus nerve damage or neurotransmitter dysfunction (nicotine is not effective in stimulating muscarinic receptors). This oral formulation is unique because it is capable of stimulating the nicotinic acetylcholine receptors on the effector organs without exceeding Upper Tolerable Limits of the ingredients, and it does so without a viable pre- or post-ganglionic vagus nerve. Shin V Y, Wu W K, Chu K M, Wong H P, Lam E K, Tai E K, Koo M W, Cho C H. Nicotine induces cyclooxygenase-2 and vascular endothelial growth factor receptor-2 in association with tumor-associated invasion and angiogenesis in gastric cancer. Mol Cancer Res. 2005 November; 3(11):607-15. doi: 10.1158/1541-7786.MCR-05-0106. PMID: 16317086.

The present invention can be used to treat damaged endothelium to decrease the risk of thromboses, leaky vessels, and atherosclerotic changes—all of which are common in connective tissue disease and autonomic dysfunction. In patients with Postural Orthostatic Tachycardia Syndrome (POTS), inflammation damaging the vascular endothelium contributes to venous pooling. This form of endotheliopathy involving, but not limited to the veins, responds to treatment with the compounds taught herein. Gualtierotti R, Ingegnoli F, Griffini S, Grovetti E, Borghi M O, Bucciarelli P, Meroni P L, Cugno M. Detection of early endothelial damage in patients with Raynaud's phenomenon. Microvasc Res. 2017 September; 113:22-28. doi: 10.1016/j.mvr.2017.04.004. Epub 2017 Apr. 25. PMID: 28450106.

A dosage unit for use of the composition of the present invention may be a single compound or mixtures thereof with other compounds. The compounds may be mixed together, form ionic or even covalent bonds. The composition of the present invention may be administered in oral, intravenous (bolus or infusion), intraperitoneal, subcutaneous, transdermal, transcutaneous, intrapulmonary, intranasal, suppositories, or intramuscular form, including prenatally, all using dosage forms well known to those of ordinary skill in the pharmaceutical arts (the only exception is that use of nicotine needs to be transdermal, vaginal suppository or anal suppository). Depending on the particular location or method of delivery, different dosage forms, e.g., tablets, capsules, pills, powders, granules, liquids, elixirs, tinctures, suspensions, syrups, and emulsions may be used to provide the composition of the present invention to a patient in need of therapy for a medical condition or symptom. The composition may also be administered as any one of known salt forms. Note that nicotine should only be delivered as a transdermal, or vaginal or anal suppository.

The composition of the present invention is typically administered in a mixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the composition may be formulated to provide, e.g., maximum and/or consistent dosing for the particular form for oral, vaginal, rectal, topical, transdermal, subcutaneous, intravenous injection or parenteral administration. While the composition may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: 49; 50; 51; 52; 53; 54; 55; all of which are incorporated by reference, and the like, relevant portions incorporated herein by reference.

For example, the composition may be included in a tablet or capsule. Tablets or capsules may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with a non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The composition may be administered in the form of liposome delivery systems, e.g., small unilamellar vesicles, large unilamallar vesicles, and multilamellar vesicles, whether charged or uncharged. Liposomes may include one or more: phospholipids (e.g., cholesterol), stearylamine and/or phosphatidylcholines, mixtures thereof, and the like.

The composition may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the composition may be coupled one or more biodegradable polymers to achieve controlled release of the composition, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the composition and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

For direct delivery to the nasal passages, sinuses, mouth, throat, esophagus, trachea, lungs and alveoli, the composition (excepting nicotine) may also be delivered as an intranasal form via use of a suitable intranasal vehicle. For dermal and transdermal delivery, the composition may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for administration of composition may include the following forms.

Capsules. Capsules may be prepared by filling standard two-piece hard gelatin capsules each with 1, 5, 10, 20, 30, 40, 50, 60, 70, 80, 90, 100, 150, 200, 250, 270, 300, 350, 400, 450, or 500 milligrams of powdered active ingredient (e.g., the composition can be taken QD, BID, or TID, the daily dose may comprise: the composition comprises the two or more active agents selected from: a choline compound selected from at least one of choline at 1 mg to 2,000 mg, lecithin at 1 mg to 4 grams, or L-alpha glycerylphosphorylcholine at 0.1 mg to 2,400 mg; 1 mcg to 50 mg of the cholinergic agonist is selected from cevimeline, carbamoylcholine, bethanechol, pilocarpine, varenicline, acetylcholine, arecoline, lobeline, GTS-21, or a nicotine patch (patch dose is 1 mg-21 mg); 1 mcg to 50 mg of the acetylcholinesterase inhibitor (or cholinesterase inhibitor) is selected from carbamates, physostigmine, neostigmine, pyridostigmine, ambenonium, demecarium, rivastigmine, phenanthrene derivatives, galantamine, caffeine (dose 1 mg-500 mg), rosmarinic acid, alpha-pinene, piperidines, donepezil, tacrine, edrophonium, huperzine A, ladostigil, ungeremine, lactucopicrin, memantine, or acotiamide, e.g., the dose of the cholinesterase inhibitor is 0.001, 0.01, 0.1, 1, 2, 5, 10, 15, 20, 25, 30, 35, 40, or 50 mg; or 1 mg to 2,000 mg of the carnitine selected from L-carnitine, Acetyl L-carnitine, L-carnitine L-tartrate, or Propionyl-L-carnitine. The composition further comprises at least one of Thiamin, or Magnesium. The dose of the choline compound is 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 3,750, or 4,000, or a range from 30 to 2,400, 40 to 2,300, 50 to 2,000, 60 to 1,500, 70 to 1,000, 80 to 750, 90 to 2,400, 200 to 2,300, 300 to 2,200, 400 to 2,100, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000, 1,600 to 2,100, 1,700 to 2,200, 1,800 to 2,300, or 1,900 to 2,400.

The dose of the carnitine is 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 30 to 2,000, 40 to 2,300, 50 to 2,000, 60 to 1,500, 70 to 1,000, 80 to 750, 90 to 2,000, 200 to 2,000, 300 to 2,000, 400 to 2,000, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, or 1,500-2,000 mg.

The dose of Thiamin is 0.1, 0.5, 1, 5, 10, 20, 30, 40, 50, 60, 70, 80, 90, 100, 150, 200 mg. The dose of Magnesium is 0.1, 0.2, 0.3, 0.5, 0.75, 1.0, 5, 10, 15, 20, 25, 30, 40, 50, 60, 70, 80, 90, 100, 150, 200, 250, 300, 350, 400, 450, 500, 540, 600, 700, 750, or 800 mg.

Soft Gelatin Capsules. A mixture of active ingredient is dissolved in a digestible oil such as soybean oil, cottonseed oil or olive oil. The active ingredient is prepared and injected by using a positive displacement pump into gelatin to form soft gelatin capsules containing, e.g., 1, 5, 10, 20, 30, 40, 50, 60, 70, 80, 90, 100, 150, 200, 250, 270, 300, 350, 400, 450, or 500 milligrams of the active ingredient. The capsules are washed and dried.

Tablets. A large number of tablets are prepared by conventional procedures so that the dosage unit was 1, 5, 10, 20, 30, 40, 50, 60, 70, 80, 90, 100, 150, 200, 250, 270, 300, 350, 400, 450, or 500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin.

For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

For rectal and vaginal routes of administration, the composition of the present invention can be formulated as solutions, retention enemas, suppositories or ointments containing conventional suppository bases such as cocoa butter or other glycerides. Suppositories may also include about 0.5% to about 50% of a compound of the invention, disposed in a polyethylene glycol (PEG) carrier, for example, PEG 1000 (96%) and PEG 4000 (4%).

An exemplary transdermal device generally includes a reservoir defined by an impermeable backing layer and a membrane. The backing layer and the membrane are joined together about the outer periphery of the device. These layers may be joined by an adhesive, a heat seal, or the like. The transdermal device may also include an adhesive layer to attach the device to the skin of a subject. A release liner will generally cover the adhesive that the user removes prior to use of the device to expose adhesive layer.

Backing layer defines the distal side of the patch, that is, the side furthest from the skin in use. The backing layer functions as the primary structural element of the device and provides the device with its mechanical properties, e.g., flexibility. The backing layer serves as a protective, impermeable covering to prevent loss of the particles containing the active compound(s) in the reservoir. Suitable backing materials include commercially available films for medical use, such as those supplied by 3M corporation, Dow Chemical or Fasson Medical Industries. Typical backing materials are made from polyester or the like and may be pigmented or metallized.

The reservoir is defined generally by the space or gap between the backing layer and the membrane, provides a storage structure in which to retain the suspension of particles containing the active compound(s) to be administered. One side of the reservoir is generally defined by a highly porous member that retains the formulation within the reservoir, i.e., it deters bulk flow of the formulation out of the reservoir, but allows passage of the formulation from the reservoir into the skin. Materials suitable for use as membrane include non-woven fabrics such as nonwoven polyesters, polyethylene, polypropylene and other synthetic polymers. The material is heat or otherwise sealable to the backing layer to provide a barrier to transverse flow of reservoir contents.

Adhesive layer is the means by which the device is affixed to the skin. This layer is made from a pharmaceutically acceptable pressure sensitive adhesive, such as polydimethylsiloxane, polyisobutylene, polyacrylate, polyurethane and the like. It will be appreciated that the adhesive layer can also be a peripheral, or rim, adhesive layer.

The transdermal device containing the particles containing active compound(s) may also include a peel strip or release liner to cover the surface of the adhesive layer and to prevent loss of reservoir contents during storage. Prior to use, the release liner is removed from the device. The release liner is typically a material impermeable to the reservoir contents, for example polyethylene terephthalate, and is releasable usually by treatment with a silicone or fluorocarbon.

Transdermal devices generally include a backing layer, a membrane and a peripheral adhesive layer. The backing layer and membrane may be glued or heat-sealed about the periphery of the device. A reservoir defined by the space between the backing layer and the membrane provides for storage of particles containing the active compound(s) to be administered transdermally. The peripheral adhesive layer may be applied directly to backing layer. A release liner protects the device during storage.

The contents of the reservoir may even be in direct contact with the skin when the device is affixed to a subject. The reservoir in this device is composed of an absorbent sponge or a porous, highly permeable polymer. Materials suitable for the reservoir include polyurethane, polyethylene or polypropylene materials. An impermeable backing layer prevents loss of reservoir contents through the distal, top side of the device. The backing layer is coated on its distal side with an adhesive overlay, which is protected by a backing or polymer layer. Prior to use, the peripheral edge of the adhesive overlay is exposed by peeling a release liner and an impermeable protective strip from the proximal, skin side of the device. The transdermal delivery device may be adhesively attached to the skin of the user, although other methods for attaching the device to the skin are contemplated and suitable, e.g., an elastic arm band or an adjustable belt.

Transdermal device membranes are generally porous, highly permeable membranes with minimal resistance to diffusion of the reservoir contents, relative to the skin. At the same time, the membrane functions to prevent bulk flow of the particles containing the active compound(s) in the reservoir. Materials suitable for use as a membrane include hydrophilic and hydrophobic fabrics, cloths and polymer films having a porosity suitable for retaining the particles containing the active compound(s). Such materials may be nonwoven or woven, yet having a defined pore size. It will be appreciated that the membrane can be selected to provide more or less diffusional resistance as desired. For example, to design a device where the membrane is rate controlling, rather than the skin, a membrane with a tighter weave or smaller pore size can be selected.

Kits. The present invention also includes pharmaceutical kits useful, for example, for the treatment of medical conditions associated with the diseases discussed hereinabove, which comprise one or more containers containing a pharmaceutical composition comprising a therapeutically effective amount of the components of the composition. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

Patient Case Studies.

Female, 45 years old diagnosed with hypermobile Ehlers-Danlos syndrome suffered with chronic constipation and fatigue. A physician, she underwent extensive testing for an underlying medical cause of her symptoms and all testing results were normal. Fatigue continued until she was unable to come to work most days. A nicotine patch (10 mg) placed on her abdomen relieved her constipation—she had a bowel movement 90 minutes after placement (there was no relief of fatigue). The nicotine patch was removed and constipation returned. The oral compound of the present invention (600 mg Alpha-GPC with 75 mcg Huperzine A) resulted in a bowel movement 90 minutes after ingesting it. The patient expressed amazement because she even experienced a bowel movement after eating sushi, which she said “never happens”. Importantly, her fatigue was also eliminated, allowing her to return to work full-time.

Female, 17 years old with significant visual snow was forced to discontinue playing volleyball (she received a scholarship for volleyball) because the visual snow significantly limited her ability to see. She was found to have POTS—Postural Orthostatic Tachycardia Syndrome (her heart rate increased more than 30 bpm on a tilt table test). She also suffered with poor digestion, dry eyes, and fatigue. She was found to have Idiopathic Intracranial Hypertension (pseudotumor cerebri) and acetazolamide (250 mg three times per day) relieved her pseudotumor cerebri symptoms but did not improve her visual snow. Two weeks after beginning use of the compound taught herein (400 mg Alpha GPC, 150 mg Carnitine, and 10 mg Thiamin), her visual snow was “95% gone” and she was able to return to playing volleyball. Her digestion normalized and her dry eyes and fatigue also resolved.

Female, 50 years old diagnosed with Ehlers-Danlos syndrome was given Vancomycin for a hospital-induced infection and developed hives and dramatic flushing (“red man's syndrome”). In approximately 4 days, she developed steatorrhea, with straw-colored stool and water on the top of the toilet bowel. She was too ill for an evaluation of pancreatitis and instead was put on the oral compound of the present invention (2000 mg Choline and 30 mg Pyridostigmine, taken TID). Within days, stool color began to normalize and was completely normal in one month.

Female, 48 years old, had suffered for three years with poor digestion and her stool looked like food that had gone into a blender (there was no form to her stools, and food was clearly visible). The patient was diagnosed with Joint Hypermobility Syndrome and dysautonomia with tachycardia. Pancreatic enzyme deficiency was suspected. The oral compound of the present invention (150 mg Acetyl-L-Carnitine, 2000 mg Lecithin) resulted in normally formed stool (brown and banana shaped) within days and remained a normal consistency as long as she ingested the oral compound daily.

Five patients (three female, two male) diagnosed with POTS (Postural Orthostatic Tachycardia Syndrome) and gastroparesis got relief from gastroparesis within two hours of a nicotine patch being placed on their abdomens (the two males used 21 mg patch, the women were provided with 10.5 mg patch). All successfully switched from using a nicotine patch to taking the oral compound of the present invention (600 mg Alpha GPC, 150 mg Acetyl-L-Carnitine, and 75 mcg Huperzine) and had normal, daily bowel movements.

Female, 21 years old, was diagnosed with a connective tissue disorder (Joint Hypermobility Syndrome) and POTS (Postural Orthostatic Tachycardia Syndrome). Most every day (usually in the evenings) she experienced pseudo seizures lasting from 30 minutes to three hours. These began with her eyes rolling up and her eyelids fluttering, then they progressed to full body twitching and spasming. These pseudo seizures continued for over three years, but physicians were unable to locate the source of her symptoms. A Roth spot in her ocular fundus in addition to numerous symptoms of nutrient malabsorption were noted. She suffered with light sensitivity secondary to large pupils (5.5 mm), weight loss, extreme fatigue, constipation, tachycardia, and dry eyes. The oral compound of the present invention (600 mg Alpha-GPC with 75 mcg Huperzine A), when combined with Vitamin B6, stopped her pseudo seizures within two days, and her constipation was relieved. Her fatigue diminished and normalized weeks later. Her tachycardia, dry eyes, and light sensitivity also went away. The Roth spot went away within 4 days and never returned.

Female, 52 years old was diagnosed with Ehlers-Danlos syndrome and dysautonomia. She suffered with extreme mental fatigue (she was unable to make a list of even two things to accomplish), orthostatic intolerance, tachycardia (resting heart rate was 123 bpm), constipation (no bowel movement for 11 days), and low gallbladder function (HIDA scan revealed an ejection fraction of 18%). Gallbladder removal surgery was recommended. Fluoroscopy revealed ileocecal valve dysfunction. A 21 mg nicotine patch placed on the abdomen relieved her constipation within 90 minutes. The oral compound of the present invention (400 mg Alpha GPC, 150 mg Carnitine, and 10 mg Thiamin) resulted in normal bowel movements and a return of gallbladder function. Tachycardia was relieved as well as extreme mental fatigue.

A 40-year-old female diagnosed with Postural Orthostatic Tachycardia Syndrome (POTS) suffered with visual snow, fatigue, high intracranial pressure, and constipation. The oral compound of the present invention (400 mg Alpha GPC, 150 mg Carnitine, and 10 mg Thiamin) when taken daily, resulted in normal bowel movements. Acetazolamide was prescribed for high intracranial pressure. In four weeks, her visual snow was dramatically improved, her fatigue was improving, and constipation never returned.

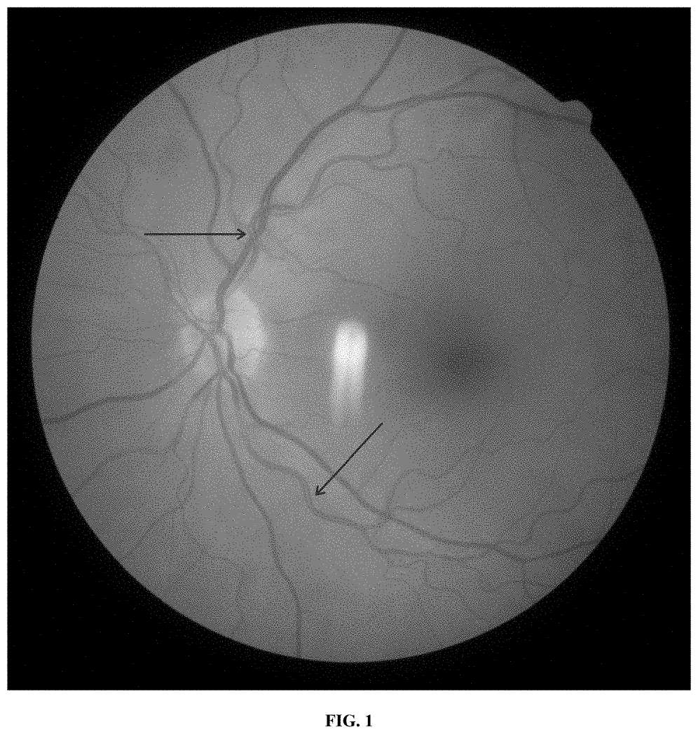

FIG. 1 shows blue arrows that point to premature plaque formation in arterioles with narrowed vascular lumen.

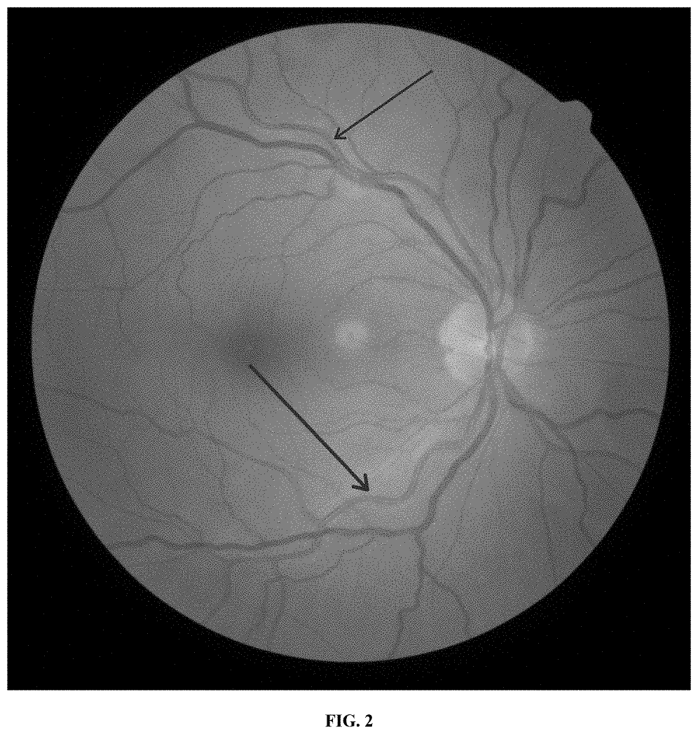

FIG. 2 shows blue arrows that point to premature plaque formation in arterioles with narrowed vascular lumen.

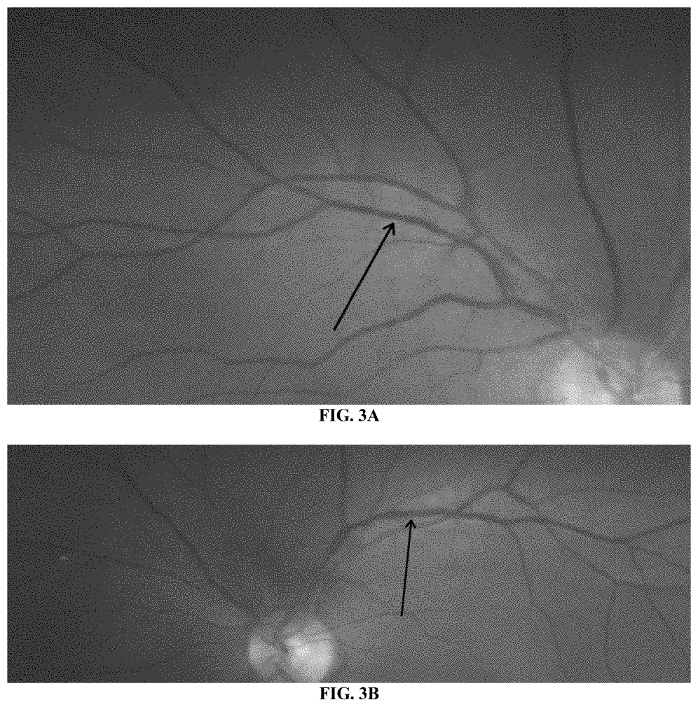

FIGS. 3A and 3B show black arrows that point to venous fibrosis evidenced by irregular caliber.

Other recipes of the present invention include:

A tablet or capsule (e.g., a vegetarian capsule) comprising: at least one of choline, lecithin, or L-Alpha Glycerylphosphorylcholine (“Alpha GPC”): 600 mg (e.g., if 50% elemental), Acetyl-L-Carnitine: 300 mg, Huperzine A: 150 mcg, and optionally 10-30 mg Thiamin and/or 0.3 to 100 mg Magnesium. In certain examples the dosage for can be halved and provided in a dosage of two capsules per dosage, taken 1-3 times per day.

Nicotine, e.g., 1 to 21 mg nicotine patch can be used alone, for short periods of time (if the patients do not exhibit skin irritation, skin welting and/or dermatographia) to test the viability of the post-ganglionic nicotinic acetylcholinergic receptors and/or effector organs, or for short-term treatment, especially useful in ileocecal valve dysfunction and gastroparesis

The composition may also include two or more agents selected from: a choline compound selected from at least one of choline compound is 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 3,750, or 4,000, or a range from 30 to 2,400, 40 to 2,300, 50 to 2,000, 60 to 1,500, 70 to 1,000, 80 to 750, 90 to 2,400, 200 to 2,300, 300 to 2,200, 400 to 2,100, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000, 1,600 to 2,100, 1,700 to 2,200, 1,800 to 2,300, or 1,900 to 2,400, the cholinergic agonist is selected from cevimeline, carbamoylcholine, bethanechol, pilocarpine, varenicline, acetylcholine, arecoline, lobeline, GTS-21, or a nicotine patch (patch dose is 1 mg-21 mg); 1 mcg to 50 mg of the acetylcholinesterase inhibitor (or cholinesterase inhibitor) is selected from carbamates, physostigmine, neostigmine, pyridostigmine, ambenonium, demecarium, rivastigmine, phenanthrene derivatives, galantamine, caffeine (dose 1 mg-500 mg), rosmarinic acid, alpha-pinene, piperidines, donepezil, tacrine, edrophonium, huperzine A, ladostigil, ungeremine, lactucopicrin, memantine, or acotiamide, e.g., the dose of the cholinesterase inhibitor is 0.001, 0.01, 0.1, 1, 2, 5, 10, 15, 20, 25, 30, 35, 40, or 50 mg; carnitine is 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 30 to 2,000, 40 to 2,300, 50 to 2,000, 60 to 1,500, 70 to 1,000, 80 to 750, 90 to 2,000, 200 to 2,000, 300 to 2,000, 400 to 2,000, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, or 1,500-2,000 mg and optionally Thiamin is 0.1, 0.5, 1, 5, 10, 20, 30, 40, 50, 60, 70, 80, 90, 100, 150, 200 mg. The dose of Magnesium is 0.1, 0.2, 0.3, 0.5, 0.75, 1.0, 5, 10, 15, 20, 25, 30, 40, 50, 60, 70, 80, 90, 100, 150, 200, 250, 300, 350, 400, 450, 500, 540, 600, 700, 750, or 800 mg.

A dose, e.g., a tablet, capsule or liquid, that includes two or more agents selected from: a choline compound selected from at least one of choline compound is 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 3,750, or 4,000, or a range from 30 to 2,400, 40 to 2,300, 50 to 2,000, 60 to 1,500, 70 to 1,000, 80 to 750, 90 to 2,400, 200 to 2,300, 300 to 2,200, 400 to 2,100, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000, 1,600 to 2,100, 1,700 to 2,200, 1,800 to 2,300, or 1,900 to 2,400, the cholinergic agonist is selected from cevimeline, carbamoylcholine, bethanechol, pilocarpine, varenicline, acetylcholine, arecoline, lobeline, GTS-21, or a nicotine patch (patch dose is 1 mg-21 mg); 1 mcg to 50 mg of the acetylcholinesterase inhibitor (or cholinesterase inhibitor) is selected from carbamates, physostigmine, neostigmine, pyridostigmine, ambenonium, demecarium, rivastigmine, phenanthrene derivatives, galantamine, caffeine (dose 1 mg-500 mg), rosmarinic acid, alpha-pinene, piperidines, donepezil, tacrine, edrophonium, huperzine A, ladostigil, ungeremine, lactucopicrin, memantine, or acotiamide, e.g., the dose of the cholinesterase inhibitor is 0.001, 0.01, 0.1, 1, 2, 5, 10, 15, 20, 25, 30, 35, 40, or 50 mg; carnitine is 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 30 to 2,000, 40 to 2,300, 50 to 2,000, 60 to 1,500, 70 to 1,000, 80 to 750, 90 to 2,000, 200 to 2,000, 300 to 2,000, 400 to 2,000, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, or 1,500-2,000 mg and optionally Thiamin is 0.1, 0.5, 1, 5, 10, 20, 30, 40, 50, 60, 70, 80, 90, 100, 150, 200 mg. The dose of Magnesium is 0.1, 0.2, 0.3, 0.5, 0.75, 1.0, 5, 10, 15, 20, 25, 30, 40, 50, 60, 70, 80, 90, 100, 150, 200, 250, 300, 350, 400, 450, 500, 540, 600, 700, 750, or 800 mg in a suitable non-active excipient.

A composition reflecting the complete daily dosage consisting essentially of: two or more agents selected from: a choline compound selected from at least one of choline compound is 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 3,750, or 4,000, or a range from 30 to 2,400, 40 to 2,300, 50 to 2,000, 60 to 1,500, 70 to 1,000, 80 to 750, 90 to 2,400, 200 to 2,300, 300 to 2,200, 400 to 2,100, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000, 1,600 to 2,100, 1,700 to 2,200, 1,800 to 2,300, or 1,900 to 2,400, the cholinergic agonist is selected from cevimeline, carbamoylcholine, bethanechol, pilocarpine, varenicline, acetylcholine, arecoline, lobeline, GTS-21, or a nicotine patch (patch dose is 1 mg-21 mg); 1 mcg to 50 mg of the acetylcholinesterase inhibitor (or cholinesterase inhibitor) is selected from carbamates, physostigmine, neostigmine, pyridostigmine, ambenonium, demecarium, rivastigmine, phenanthrene derivatives, galantamine, caffeine (dose 1 mg-500 mg), rosmarinic acid, alpha-pinene, piperidines, donepezil, tacrine, edrophonium, huperzine A, ladostigil, ungeremine, lactucopicrin, memantine, or acotiamide, e.g., the dose of the cholinesterase inhibitor is 0.001, 0.01, 0.1, 1, 2, 5, 10, 15, 20, 25, 30, 35, 40, or 50 mg; carnitine is 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 30 to 2,000, 40 to 2,300, 50 to 2,000, 60 to 1,500, 70 to 1,000, 80 to 750, 90 to 2,000, 200 to 2,000, 300 to 2,000, 400 to 2,000, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, or 1,500-2,000 mg and optionally Thiamin is 0.1, 0.5, 1, 5, 10, 20, 30, 40, 50, 60, 70, 80, 90, 100, 150, 200 mg. The dose of Magnesium is 0.1, 0.2, 0.3, 0.5, 0.75, 1.0, 5, 10, 15, 20, 25, 30, 40, 50, 60, 70, 80, 90, 100, 150, 200, 250, 300, 350, 400, 450, 500, 540, 600, 700, 750, or 800 mg in a suitable non-active excipient.

A composition reflecting the complete daily dosage consisting of: two or more agents selected from: a choline compound selected from at least one of choline compound is 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 3,750, or 4,000, or a range from 30 to 2,400, 40 to 2,300, 50 to 2,000, 60 to 1,500, 70 to 1,000, 80 to 750, 90 to 2,400, 200 to 2,300, 300 to 2,200, 400 to 2,100, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000, 1,600 to 2,100, 1,700 to 2,200, 1,800 to 2,300, or 1,900 to 2,400, the cholinergic agonist is selected from cevimeline, carbamoylcholine, bethanechol, pilocarpine, varenicline, acetylcholine, arecoline, lobeline, GTS-21, or a nicotine patch (patch dose is 1 mg-21 mg); 1 mcg to 50 mg of the acetylcholinesterase inhibitor (or cholinesterase inhibitor) is selected from carbamates, physostigmine, neostigmine, pyridostigmine, ambenonium, demecarium, rivastigmine, phenanthrene derivatives, galantamine, caffeine (dose 1 mg-500 mg), rosmarinic acid, alpha-pinene, piperidines, donepezil, tacrine, edrophonium, huperzine A, ladostigil, ungeremine, lactucopicrin, memantine, or acotiamide, e.g., the dose of the cholinesterase inhibitor is 0.001, 0.01, 0.1, 1, 2, 5, 10, 15, 20, 25, 30, 35, 40, or 50 mg; carnitine is 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 30 to 2,000, 40 to 2,300, 50 to 2,000, 60 to 1,500, 70 to 1,000, 80 to 750, 90 to 2,000, 200 to 2,000, 300 to 2,000, 400 to 2,000, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, or 1,500-2,000 mg and optionally Thiamin is 0.1, 0.5, 1, 5, 10, 20, 30, 40, 50, 60, 70, 80, 90, 100, 150, 200 mg. The dose of Magnesium is 0.1, 0.2, 0.3, 0.5, 0.75, 1.0, 5, 10, 15, 20, 25, 30, 40, 50, 60, 70, 80, 90, 100, 150, 200, 250, 300, 350, 400, 450, 500, 540, 600, 700, 750, or 800 mg in a suitable delivery dose.

Discussion of Ingredients:

Choline compounds may include choline at the amounts as set forth hereinabove, e.g., 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, lecithin at the amounts as set forth hereinabove, e.g., 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 3,750, or 4,000 mg, or Alpha Glycerylphosphorylcholine at the amounts as set forth hereinabove, e.g., 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 2,100, 2,200, 2,300, or 2,400 mg (also known as “Alpha-GPC” or “choline alfoscerate”), which is a natural choline compound and is a parasympathomimetic acetylcholine precursor. Alpha-GPC is derived from highly purified soy lecithin and is a biosynthetic precursor of the acetylcholine neurotransmitter (contributing the “choline” component). The ingestion of choline as a mono-ingredient was insufficient to stimulate the effector organs of the vagus nerve. When in sufficient quantity, choline when combined with one additional ingredient, is effective in triggering the effector organs of the vagus nerve (much as a sudden burst of transdermal nicotine will accomplish). Alpha-GPC can be taken in much smaller quantities than choline or lecithin and is less likely to result in gastrointestinal side-effects. If the same ingredients are taken in small quantities throughout the day, the receptors on the effector organs will not respond in the same way (triggering of the organ to respond). If the compound is taken in the morning (preferably on an empty stomach), the reassurance of vagus nerve effector organ is achieved when a bowel movement occurs within 90 minutes of ingestion. When small doses are taken throughout the day, a bowel movement is not an immediate consequence of ingestion, and assurance of vagus nerve stimulation is not possible. Therefore, dosing and timing of the compound is critical. Most importantly, this compound does not require a functioning pre- or post-ganglionic vagus nerve to be effective (the presence of a viable receptor on the effector organ is sufficient), regardless of the patient's dietary requirements or genetic polymorphisms.

Not intending to be bound by theory, and in no way a limitation of the present invention, carnitine supplementation is known to be beneficial in patients with chronic fatigue (a co-morbid presentation of most, if not all “autoimmune” disorders). These patients have been shown to have low levels of carnitine in their serum. Although likely multi-factorial, in the case studies performed, patients with organ malfunction secondary to poor vagus nerve function invariably achieved better organ function when carnitine was added to the compound. Another reason for the success of Acetyl-L-Carnitine, when used specifically, is because it acts as a precursor of acetylcholine, through the use of Carnitine acetyltransferase, rather than Choline acetyltransferase for the manufacture of acetylcholine.

Cholinesterase inhibitors increase the levels of acetylcholine in the synapse by slowing its breakdown by suppressing cholinesterase. Although insufficient by itself to stimulate the effector organs of the vagus nerve, when combined with any other ingredient of the compound, stimulation occurs. One reason that the use of cholinesterase inhibitors is not as effective by itself, is that these slow the breakdown of acetylcholine, but they do not allow increased release of acetylcholine, they do not workaround genetic disorders of acetylcholine production, nor do they include ingredients that could be necessary for the production of acetylcholine. When inflammatory agents are blocking the release of acetylcholine, or when the pre- or post-ganglionic nerves are damaged, cholinesterase inhibitors when in combination with an additional ingredient of the compound result in triggering of the effector organ. Huperzine A is a compound found in the plant Huperzia serrata and is a potent acetylcholinesterase inhibitor. It is capable of crossing the blood-brain barrier and can also preserve the acetylcholine released in the central nervous system. Cholinesterase inhibitors may also decrease norepinephrine levels, which are high in the hyperadrenergic postural orthostatic tachycardia syndrome patient, further assisting recovery from autonomic dysfunction.

Nicotine is a powerful acetylcholinergic agonist for the vagus nerve and is found in the leaves of the tobacco plant (Nicotiana tabacum). Transdermal nicotine (1-21 mg) was found to uniquely stimulate the nicotinic acetylcholinergic receptors on the organs (it did not depend upon the presence of a pre- or post-ganglionic vagus nerve), thus enhancing parasympathetic neurotransmission. The dose must be sufficient for response, but not so high that it antagonizes the nicotinic receptors (and results in the Bezold-Jarisch reflex). A dosage of 60 mg is lethal. In patients tested, oral nicotine tended to increase symptoms of orthostatic intolerance (such as tachycardia and hypotension) whereas transdermal nicotine (1-21 mg) never failed to trigger the effector organ. Thus, the use of transdermal nicotine can be used as a diagnostic tool to test the efficacy of the receptors at the effector organs controlled by the vagus nerve. A functioning vagus nerve is not necessary for response. When effective, treatment with the compound is effective at stimulating the effector organ without the negative consequences of nicotine.

Thiamin (also known as Thiamine or Vitamin B-1) deficiency is commonly seen in patients with autonomic dysfunction and/or connective tissue disorders as a tardive consequence of disrupted bowel and organ function. Blitshteyn S. Vitamin B1 deficiency in patients with postural tachycardia syndrome (POTS). Neurol Res. 2017 August; 39(8):685-688. doi: 10.1080/01616412.2017.1331895. Epub 2017 May 21. PMID: 28531358. Thiamin levels can also drop due to a diet low in thiamin, or in conditions such as chronic diarrhea, anorexia, alcoholism and in patients taking medications including, but not limited to diuretics, quercetin and rutin, and/or in patients low in transketolase. Thiamin may be provided in any of the following forms: Allithiamine, aneurine, aneurine HCl, aneurine mononitrate, antiberiberi factor, antiberiberi vitamin, antineuritic factor, antineuritic vitamin, anurine, B-complex vitamin, benfotiamine, beta-hydroxy-ethylthiazolium chloride, sulfotiamine, thiamin, thiamin chloride, thiamin diphosphate, thiamin HCl, thiamin hydrochloride, thiamin monophosphate (TMP), thiamin nitrate, thiamin pyrophosphate (TPP), thiamin triphosphate (TTP), thiamine, thiamine chloride, thiamine diphosphate, thiamine HCl, thiamine hydrochloride, thiamine monophosphate (TMP), thiamine nitrate, thiamine pyrophosphate (TPP), thiamine tetrahydrofurfuryl disulfide, thiamine triphosphate (TTP), thiaminium chloride HCl or thiaminium chloride hydrochloride.

Supplementation with Thiamin alone will not eliminate autonomic dysfunction or disorders of the effector organs of the parasympathetic nervous system. The compound must include any two of the following: a choline compound, an anticholinesterase compound, a cholinergic agonist, or a carnitine to stimulate organ function. Magnesium can be added to assist in Thiamin absorption.

Magnesium supplementation may be in one or more of the various forms available, including but not limited to: Magnesium chloride, oxide, gluconate, malate, orotate, glycinate, L-threonate, and citrate. Magnesium deficiency contributes to thiamin deficiency, which can ultimately result in the low production of acetylcholine, contributing to autonomic dysfunction.

Not intending to be bound by theory, and in no way a limitation of the present invention, the invention can be used as an addition to a normally prescribed pre-natal vitamin in order to prevent vascular abnormalities, such as inadequate veins and chronic cerebrospinal venous insufficiency (which can also result in any form of increased intracranial hypertension), through moderation of angiogenesis in the fetal brain and in vessels draining the brain. Such moderation of angiogenesis has proven to be critical in the development of the hypothalamus. Studies show that patients with connective tissue disorders and vascular disorders exhibit abnormal amygdala, which can affect memory from childhood, and continue throughout the patient's life. This results in a decline in spatial relationship and proprioceptive representations. Previous studies in rats showed that maternal dietary choline supplementation increases the size of the cell body of cholinergic neurons and decreases choline acetyltransferase activity in brain, whereas choline deficiency decreases cholinergic activity in the offspring's brain. The embryologic timing of neurogenesis results in deficiencies in spatial learning, long-term memory and temporal processing. Because these abnormalities are permanent, prevention of such vascular and neurological abnormalities must be avoided via treatment of the mother throughout her pregnancy. The addition of choline is not sufficient when the mother has poor gastrointestinal absorption of nutrients (as is seen in autonomic dysfunction, for example). The compound including any two of the following of a choline compound, a cholinergic agonist, an anticholinesterase compound and carnitine is needed to stimulate proper organ function (and therefore normal nutrient absorption) via stimulation of the effector organs of the vagus nerve. The composition and method of use of the present invention is unique because it corrects for numerous causes of autonomic dysfunction and includes ingredients in the quantities and form required to work around genetic, vascular, and/or co-morbid components involved.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context. In certain embodiments, the present invention may also include methods and compositions in which the transition phrase “consisting essentially of” or “consisting of” may also be used.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Traber G L, Piccirelli M, Michels L. Visual snow syndrome: a review on diagnosis, pathophysiology, and treatment. Curr Opin Neurol. 2020 February; 33(1):74-78. doi: 10.1097/WCO.0000000000000768. PMID: 31714263.

What is claimed is:

1. A composition comprising:
at least two active agents selected from: a choline compound; a cholinergic agonist; a cholinesterase inhibitor; and a carnitine, in an amount effective to treat at least one of a connective tissue disorder, compression of, damage to, or infection of, any portion of a preganglionic or postganglionic vagus nerve, autonomic dysfunction, autonomic neuropathy with partially functioning cholinergic receptors, post-viral and post-infective autonomic dysfunction, post-traumatic autonomic dysfunction, physical trauma or mental/emotional trauma, traumatic brain injury (TBI), post-traumatic stress disorder (PTSD), a genetic disorder of an acetylcholine production cycle, inflammatory autonomic dysfunction or inflammatory Postural Orthostatic Tachycardia Syndrome (POTS), chronic infectious and/or fatigue syndromes, Chronic Fatigue Syndrome, post-COVID (“Long-haulers”) or Post-Acute sequelae of COVID-19 (Post-Acute Sequelae SARS-CoV-2 infection), Myalgic Encephalomyelitis, Chronic Lyme disease, or fibromyalgia.

2. The composition of claim 1, wherein the composition treats or prevents at least one of:
connective tissue disorder is a disease associated with collagen, fibrillin, microfibrillin, elastin, or tenascin-X (genetic, acquired, or both); the autonomic dysfunction is due to or worsened by genetic disorders of inflammation (RCCX, CYP21A2, disorders of the TGF-beta cascade, MCP-1, TNX, SMAD), autoimmune disorders, abnormal hormones (androgens, estrogens or both), mast cell activation disorders, genetic disorders of tryptase, eosinophilic disorders (eosinophilic esophagitis, eosinophilic gastroenteritis, eosinophilia), adrenal disorders, congenital adrenal hyperplasia, Hyper IgE syndrome, low immune system (low IgG₁, IgG₂, IgG₃, IgG₄, or IgA), Idiopathic Intracranial Hypertension (IIH), post-infective inflammation (post-viral, post-SARS, post-COVID, or post-bacterial); vascular inflammation, vascular oxidation, hemochromatosis, hemolysis, reduced levels of nitric oxide, or impaired release of acetylcholine; or wherein the composition treats or prevents at least one of: autonomic dysfunction (dysautonomia, Postural Orthostatic Tachycardia Syndrome); multiple organ dysfunction (constipation, gastroparesis, idiopathic gastrointestinal dysmotility, low gastric acid production, ileocecal valve dysfunction (“ileus”), breathing difficulties, low gallbladder ejection fractions, biliary dyskinesia, acalculous gallbladder disease, Sphincter of Oddi dysfunction, low cholecystokinin (“CCK”) production, poor kidney function, non-alcoholic steatohepatitis (or “NASH”), or non-alcoholic fatty liver disease); or the composition treats or prevents at least one of acquired disorders of connective tissue as seen in Ehlers-Danlos syndrome, Marfan's syndrome, Loeys-Dietz syndrome, Stickler syndrome, fibrillin disorders, elastin disorders, Joint Hypermobility Syndrome;
treats or prevents one or more signs or symptoms selected from: poor temperature control (poor thermoregulation), poor blood pressure control, large pupils, light sensitivity, poor ocular accommodation, early satiety, bloating, nausea, vomiting, difficulty swallowing, heartburn, delayed gastric emptying, loss of appetite, poor appetite regulation, diarrhea, constipation, gastric ilius, abdominal bloating, small intestine bacterial overgrowth (SIBO), small intestine fungal overgrowth (SIFO), dysbiosis, chronic candida, orthostatic hypotension, Postural Orthostatic Tachycardia Syndrome (POTS), exercise intolerance, dizziness, chronic fatigue, brain fog, diminished concentration, fainting, loss of short-term memory, loss of executive function, tachycardia, bradycardia, poor blood pressure regulation, orthostatic intolerance, anxiety, panic, panic attacks, flushing, environmental sensitivities, vascular endothelial damage, vascular leaking, dilated perivascular spaces, edema, clotting disorders, disseminated intravascular coagulation, paresthesia, visual disturbances, vision loss, visual snow, urinary retention, hallucinations (visual and auditory), disorientation, delirium, hyperactivity, restlessness, narrow angle glaucoma, muscle twitching, fasciculations, pseudoseizures, dysarthria, or poor coordination; or
treats or prevents at least one of: immune disorders, autoimmune disorders, autoinflammatory disorders, a vascular disease and a rheumatological disease, treats or prevents dry eyes, xerostomia (dry mouth), vascular endothelial disorders, or poor nitric oxide production, chronic fungal infections, or hallucinations, visual snow, prevents or restores vascular endothelial health as determined by brachial artery ultrasound or spectral-domain optical coherence tomography, decreased thrombosis, improved vascular abnormalities, arrest of venous fibrosis, elimination of orthostatic tachycardia, or reduction of livido reticularis.

3. The composition of claim 1, wherein the acetylcholinesterase inhibitor (cholinesterase inhibitor) is selected from pyridostigmine, donepezil, tacrine, galantamine, and memantine, carbamates, physostigmine, neostigmine, ambenonium, demecarium, rivastigmine, phenanthrene derivatives, galantamine, caffeine, rosmarinic acid, alpha-pinene, piperidines, tacrine, edrophonium, huperzine A, ladostigil, ungeremine, lactucopicrin, or acotiamide.

4. The composition of claim 1, wherein the cholinergic agonist is selected from cevimeline, carbamoylcholine, bethanechol, pilocarpine, varenicline, acetylcholine, arecoline, lobeline, GTS-21, or is provided as a nicotine patch.

5. The composition of claim 1, wherein the carnitine is selected from L-carnitine, Acetyl L-carnitine, L-carnitine L-tartrate, or Propionyl-L-carnitine.

6. The composition of claim 1, wherein
the two active agents are selected from: the choline compound and the cholinergic agonist; the choline compound and the acetylcholinesterase inhibitor (or cholinesterase inhibitor); the choline compound and the carnitine; the cholinergic agonist and the acetylcholinesterase inhibitor; the cholinergic agonist and the carnitine; or the acetylcholinesterase inhibitor and the carnitine;
the three active agents are selected from: the choline compound, the cholinesterase inhibitor and the cholinergic agonist; the choline compound, the acetylcholinesterase inhibitor and the carnitine; the choline, the cholinergic agonist, and the carnitine; or
the acetylcholinesterase inhibitor, the cholinergic agonist and the carnitine; or the four active agents selected from: the choline compound, the acetylcholinesterase inhibitor, the cholinergic agonist and the carnitine.

7. The composition of claim 1, wherein the composition comprises the two or more active agents selected from:
a choline compound at 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 3,750, or 4,000, or a range from 30 to 2,400, 40 to 2,300, 50 to 2,000, 60 to 1,500, 70 to 1,000, 80 to 750, 90 to 2,400, 200 to 2,300, 300 to 2,200, 400 to 2,100, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000, 1,600 to 2,100, 1,700 to 2,200, 1,800 to 2,300, or 1,900 to 2,400 mg of at least one choline;
0.001, 0.01, 0.1, 1, 2, 5, 10, 15, 20, 25, 30, 35, 40, 50 mg of the cholinesterase inhibitor selected from carbamates, physostigmine, neostigmine, pyridostigmine, ambenonium, demecarium, rivastigmine, phenanthrene derivatives, galantamine, caffeine (dose 1 mg-500 mg), rosmarinic acid, alpha-pinene, piperidines, donepezil, tacrine, edrophonium, huperzine A, ladostigil, ungeremine, lactucopicrin, memantine, or acotiamide; or
1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 30 to 2,000, 40 to 2,300, 50 to 2,000, 60 to 1,500, 70 to 1,000, 80 to 750, 90 to 2,000, 200 to 2,000, 300 to 2,000, 400 to 2,000, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000 mg of the carnitine selected from L-carnitine, Acetyl L-carnitine, L-carnitine L-tartrate, or Propionyl-L-carnitine; and
optionally comprises at least one of Thiamin, or Magnesium.

8. The composition of claim 1, wherein the dose is at least one of:
1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 3,750, or 4,000, or a range from 30 to 2,400, 40 to 2,300, 50 to 2,000, 60 to 1,500, 70 to 1,000, 80 to 750, 90 to 2,400, 200 to 2,300, 300 to 2,200, 400 to 2,100, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000, 1,600 to 2,100, 1,700 to 2,200, 1,800 to 2,300, or 1,900 to 2,400 of the choline compound;
0.001, 0.01, 0.1, 1, 2, 5, 10, 15, 20, 25, 30, 35, 40, 50 mg of the cholinesterase inhibitor; or
1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 30 to 2,000, 40 to 2,300, 50 to 2,000, 60 to 1,500, 70 to 1,000, 80 to 750, 90 to 2,000, 200 to 2,000, 300 to 2,000, 400 to 2,000, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000 mg of the carnitine; and
optionally 0.1, 0.5, 1, 5, 10, 20, 30, 40, 50, 60, 70, 80, 90, 100, 150, 200 mg Thiamin, and the range per dose is 0.1, 0.2, 0.3, 0.5, 0.75, 1.0, 5, 10, 15, 20, 25, 30, 40, 50, 60, 70, 80, 90, 100, 150, 200, 250, 300, 350, 400, 450, 500, 540, 600, 700, 750, 800 mg Magnesium.

9. The composition of claim 1, wherein the composition is adapted to be administered prenatally, orally, intravenously, intraperitoneally, intranasally, intrapulmonary, subcutaneously, intracutaneously, or intramuscularly.

10. The composition of claim 1, wherein the composition consists essentially of:
two, three, or four active agents selected from: a choline compound; a cholinergic agonist; an acetylcholinesterase inhibitor; and a carnitine; and
optionally including at least one of 0.1, 0.5, 1, 5, 10, 20, 30, 40, 50, 60, 70, 80, 90, 100, 150, 200 mg Thiamin, and the range per dose is 0.1, 0.2, 0.3, 0.5, 0.75, 1.0, 5, 10, 15, 20, 25, 30, 40, 50, 60, 70, 80, 90, 100, 150, 200, 250, 300, 350, 400, 450, 500, 540, 600, 700, 750, 800, mg Magnesium in an amount effective to treat at least one of a connective tissue disorder, compression of or damage to any portion of a preganglionic or postganglionic vagus nerve, autonomic neuropathy with partially functioning cholinergic receptors, post-viral (including post-SARS and post-COVID) and post-infective autonomic dysfunction, post-traumatic autonomic dysfunction, physical trauma or mental/emotional trauma, traumatic brain injury, post-traumatic stress disorder (PTSD), a genetic disorder of the acetylcholine manufacturing cycle, inflammatory autonomic dysfunction, or Inflammatory Postural Orthostatic Tachycardia Syndrome (POTS).

11. The composition of claim 1, wherein the composition consists of:
two, three, or four active agents selected from: a choline compound; a cholinergic agonist; an acetylcholinesterase inhibitor (cholinesterase inhibitor); and a carnitine; and
optionally including at least one of 0.1-200 mg Thiamin or 1-800 mg Magnesium in an amount effective to treat at least one of a connective tissue disorder, compression of or damage to any portion of a preganglionic or postganglionic vagus nerve, autonomic neuropathy, autonomic neuropathy with partially functioning cholinergic receptors, postganglionic vagus nerve, or both), post-viral and post-infective autonomic dysfunction, post-traumatic autonomic dysfunction, physical trauma or mental/emotional trauma, post-traumatic stress disorder (PTSD), a genetic disorder of an acetylcholine cycle, inflammatory autonomic dysfunction, or inflammatory Postural Orthostatic Tachycardia Syndrome (POTS).

12. A method of treating a disease or condition in a subject, comprising administering to a subject in need thereof an effective amount of a composition comprising at least two active agents selected from: a choline compound; a cholinergic agonist; an acetylcholinesterase inhibitor (cholinesterase inhibitor); and a carnitine, in an amount effective to treat at least one of a connective tissue disorder, compression of, damage to, or infection of, any portion of a preganglionic or postganglionic vagus nerve, autonomic dysfunction, autonomic neuropathy with partially functioning cholinergic receptors, post-viral and post-infective autonomic dysfunction, post-traumatic autonomic dysfunction, physical trauma or mental/emotional trauma, traumatic brain injury (TBI), post-traumatic stress disorder (PTSD), a genetic disorder of an acetylcholine production cycle, inflammatory autonomic dysfunction or inflammatory Postural Orthostatic Tachycardia Syndrome (POTS), chronic infectious and/or fatigue syndromes, Chronic Fatigue Syndrome, post-COVID (“Long-haulers”) or Post-Acute sequelae of COVID-19 (Post-Acute Sequelae SARS-CoV-2), Myalgic Encephalomylitis, Chronic Lyme disease, or fibromyalgia.

13. The method of claim 12, wherein
the connective tissue disorder is a disease associated with collagen, fibrillin, microfibrillin, elastin, or tenascin-X (genetic, acquired, or both);
the autonomic dysfunction is due to or worsened by genetic disorders of inflammation (RCCX, CYP21A2, disorders of the TGF-beta cascade, MCP-1, TNX, SMAD), autoimmune disorders, abnormal hormones (androgens, estrogens or both), mast cell activation disorders, genetic disorders of tryptase, eosinophilic disorders (eosinophilic esophagitis, eosinophilic gastroenteritis, eosinophilia), adrenal disorders, congenital adrenal hyperplasia, Hyper IgE syndrome, low immune system (low IgG₁, IgG₂, IgG₃, IgG₄, or IgA), Idiopathic Intracranial Hypertension (IIH), post-infective inflammation (post-viral, post-SARS, post-COVID, or post-bacterial); vascular inflammation, vascular oxidation, hemochromatosis, hemolysis, reduced levels of nitric oxide, or impaired release of acetylcholine;
treats or prevents at least one of: autonomic dysfunction (dysautonomia, Postural Orthostatic Tachycardia Syndrome); multiple organ dysfunction (constipation, gastroparesis, idiopathic gastrointestinal dysmotility, low gastric acid production, ileocecal valve dysfunction (“ileus”), breathing difficulties, low gallbladder ejection fractions, biliary dyskinesia, acalculous gallbladder disease, Sphincter of Oddi dysfunction, low cholecystokinin (“CCK”) production, poor kidney function, non-alcoholic steatohepatitis (or “NASH”), or non-alcoholic fatty liver disease;
treats or prevents at least one of acquired disorders of connective tissue as seen in Ehlers-Danlos syndrome, Marfan's syndrome, Loeys-Dietz syndrome, Stickler syndrome, fibrillin disorders, elastin disorders, Joint Hypermobility Syndrome;
treats or prevents dry eyes, xerostomia (dry mouth), vascular endothelial disorders, or poor nitric oxide production, chronic fungal infections, or hallucinations;
treats or prevents one or more signs or symptoms selected from: poor temperature control (poor thermoregulation), poor blood pressure control, large pupils, light sensitivity, poor ocular accommodation, early satiety, bloating, nausea, vomiting, difficulty swallowing, heartburn, delayed gastric emptying, loss of appetite, poor appetite regulation, diarrhea, constipation, gastric ilius, abdominal bloating, small intestine bacterial overgrowth (SIBO), small intestine fungal overgrowth (SIFO), dysbiosis, chronic candida, orthostatic hypotension, Postural Orthostatic Tachycardia Syndrome (POTS), exercise intolerance, dizziness, chronic fatigue, brain fog, diminished concentration, fainting, loss of short-term memory, loss of executive function, tachycardia, bradycardia, poor blood pressure regulation, orthostatic intolerance, anxiety, panic, panic attacks, flushing, environmental sensitivities, vascular endothelial damage, vascular leaking, dilated perivascular spaces, edema, clotting disorders, disseminated intravascular coagulation, paresthesia, visual disturbances, vision loss, visual snow, urinary retention, hallucinations (visual and auditory), disorientation, delirium, hyperactivity, restlessness, narrow angle glaucoma, muscle twitching, fasciculations, pseudoseizures, dysarthria, or poor coordination;
treats or prevents visual snow; or
prevents or restores vascular endothelial health as determined by brachial artery ultrasound or spectral-domain optical coherence tomography, decreased thrombosis, improved vascular abnormalities, arrest of venous fibrosis, elimination of orthostatic tachycardia, or reduction of livido reticularis.

14. The method of claim 12, wherein the acetylcholinesterase inhibitor (cholinesterase inhibitor) is selected from pyridostigmine, donepezil, tacrine, galantamine, and memantine, carbamates, physostigmine, neostigmine, ambenonium, demecarium, rivastigmine, phenanthrene derivatives, galantamine, caffeine, rosmarinic acid, alpha-pinene, piperidines, tacrine, edrophonium, huperzine A, ladostigil, ungeremine, lactucopicrin, or acotiamide.

15. The method of claim 12, wherein the cholinergic agonist is selected from cevimeline, carbamoylcholine, bethanechol, pilocarpine, varenicline, acetylcholine, arecoline, lobeline, GTS-21, or is provided as a nicotine patch.

16. The method of claim 12, wherein the carnitine is selected from L-carnitine, Acetyl L-carnitine, L-carnitine L-tartrate, or Propionyl-L-carnitine.

17. The method of claim 12, wherein:
the two active agents are selected from: the choline compound and the cholinergic agonist; the choline compound and the acetylcholinesterase inhibitor (or cholinesterase inhibitor); the choline compound and the carnitine; the cholinergic agonist and the acetylcholinesterase inhibitor; the cholinergic agonist and the carnitine; or the acetylcholinesterase inhibitor and the carnitine;
the three active agents are selected from: the choline compound, the cholinesterase inhibitor and the cholinergic agonist; the choline compound, the acetylcholinesterase inhibitor and the carnitine; the choline, the cholinergic agonist, and the carnitine; or the acetylcholinesterase inhibitor, the cholinergic agonist and the carnitine; or
the four active agents selected from: the choline compound, the acetylcholinesterase inhibitor, the cholinergic agonist and the carnitine.

18. The method of claim 12, wherein the composition comprises the two or more active agents selected from:
a choline compound at 1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 3,750, or 4,000, or a range from 30 to 2,400, 40 to 2,300, 50 to 2,000, 60 to 1,500, 70 to 1,000, 80 to 750, 90 to 2,400, 200 to 2,300, 300 to 2,200, 400 to 2,100, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000, 1,600 to 2,100, 1,700 to 2,200, 1,800 to 2,300, or 1,900 to 2,400 mg of at least one choline;
0.001, 0.01, 0.1, 1, 2, 5, 10, 15, 20, 25, 30, 35, 40, 50 mg of the cholinesterase inhibitor selected from carbamates, physostigmine, neostigmine, pyridostigmine, ambenonium, demecarium, rivastigmine, phenanthrene derivatives, galantamine, caffeine (dose 1 mg-500 mg), rosmarinic acid, alpha-pinene, piperidines, donepezil, tacrine, edrophonium, huperzine A, ladostigil, ungeremine, lactucopicrin, memantine, or acotiamide; or
1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 30 to 2,000, 40 to 2,300, 50 to 2,000, 60 to 1,500, 70 to 1,000, 80 to 750, 90 to 2,000, 200 to 2,000, 300 to 2,000, 400 to 2,000, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000 mg of the carnitine selected from L-carnitine, Acetyl L-carnitine, L-carnitine L-tartrate, or Propionyl-L-carnitine; and
optionally further comprises at least one of Thiamin, or Magnesium.

19. The method of claim 18, wherein the dose is at least one of:
1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 3,750, or 4,000, or a range from 30 to 2,400, 40 to 2,300, 50 to 2,000, 60 to 1,500, 70 to 1,000, 80 to 750, 90 to 2,400, 200 to 2,300, 300 to 2,200, 400 to 2,100, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000, 1,600 to 2,100, 1,700 to 2,200, 1,800 to 2,300, or 1,900 to 2,400 of the choline compound;
0.001, 0.01, 0.1, 1, 2, 5, 10, 15, 20, 25, 30, 35, 40, 50 mg of the cholinesterase inhibitor;
1, 5, 10, 20, 30, 40, 50, 60, 70, 80, 90, 100, 150, 200, 250, 270, 300, 350, 400, 450, 500, 540, 600, 700, 750, 800, 900, 1,000, 1,100, 1,200, 1,300, 1,400, 1,500, 1,600, 1,700, 1,800, 1,900, 2,000, 30 to 2,000, 40 to 2,300, 50 to 2,000, 60 to 1,500, 70 to 1,000, 80 to 750, 90 to 2,000, 200 to 2,000, 300 to 2,000, 400 to 2,000, 500 to 2,000, 600 to 1,900, 700 to 1,800, 800 to 1,700, 900 to 1,600, 1,000 to 1,500, 1,100 to 1,400, 1,200 to 1,300, 1,300 to 1,500, 1,500-2,000 mg of the carnitine; and optionally
the range per dose is 0.1, 0.5, 1, 5, 10, 20, 30, 40, 50, 60, 70, 80, 90, 100, 150, 200 mg Thiamin, and the range per dose is 0.1, 0.2, 0.3, 0.5, 0.75, 1.0, 5, 10, 15, 20, 25, 30, 40, 50, 60, 70, 80, 90, 100, 150, 200, 250, 300, 350, 400, 450, 500, 540, 600, 700, 750, 800, mg Magnesium.

20. The method of claim 12, wherein the composition is adapted to be administered prenatally, orally, intravenously, intraperitoneally, intranasally, intrapulmonary, subcutaneously, intracutaneously, or intramuscularly.

21. The method of claim 12, wherein the composition consists essentially of:
two, three, or four active agents selected from: a choline compound; a cholinergic agonist; an acetylcholinesterase inhibitor; and a carnitine; and
optionally including at least one of 0.1, 0.5, 1, 5, 10, 20, 30, 40, 50, 60, 70, 80, 90, 100, 150, 200 mg Thiamin, and the range per dose is 0.1, 0.2, 0.3, 0.5, 0.75, 1.0, 5, 10, 15, 20, 25, 30, 40, 50, 60, 70, 80, 90, 100, 150, 200, 250, 300, 350, 400, 450, 500, 540, 600, 700, 750, 800, mg Magnesium in an amount effective to treat at least one of a connective tissue disorder, compression of or damage to any portion of a preganglionic or postganglionic vagus nerve, autonomic neuropathy with partially functioning cholinergic receptors, post-viral (including post-SARS and post-COVID) and post-infective autonomic dysfunction, post-traumatic autonomic dysfunction, physical trauma or mental/emotional trauma, traumatic brain injury, post-traumatic stress disorder (PTSD), a genetic disorder of the acetylcholine manufacturing cycle, inflammatory autonomic dysfunction, or Inflammatory Postural Orthostatic Tachycardia Syndrome (POTS).

22. The method of claim 12, wherein the composition consists of:
two, three, or four active agents selected from: a choline compound; a cholinergic agonist; an acetylcholinesterase inhibitor (cholinesterase inhibitor); and a carnitine; and
optionally including at least one of 0.1-200 mg Thiamin or 1-800 mg Magnesium in an amount effective to treat at least one of a connective tissue disorder, compression of or damage to any portion of a preganglionic or postganglionic vagus nerve, autonomic neuropathy, autonomic neuropathy with partially functioning cholinergic receptors, post-viral and post-infective autonomic dysfunction, post-traumatic autonomic dysfunction, physical trauma or mental/emotional trauma, post-traumatic stress disorder (PTSD), a genetic disorder of an acetylcholine cycle, inflammatory autonomic dysfunction, or inflammatory Postural Orthostatic Tachycardia Syndrome (POTS).

23. A method for diagnosis and treatment of a compression of or damage to any portion of a preganglionic or postganglionic vagus nerve, or a post-viral and post-infective autonomic dysfunction, in a subject comprising:
identifying a subject that has one or more symptoms associated with compression of or damage to any portion of a preganglionic or postganglionic vagus nerve, or a post-viral and post-infective autonomic dysfunction, selected from at least one of: constipation, gastroparesis, idiopathic gastrointestinal dysmotility, ileocecal valve dysfunction (“ileus”), low stomach acid production, low gallbladder ejection fractions, pancreatic enzyme deficiency, tachycardia, or low acetylcholine release is suspected based on symptoms of anticholinergic syndrome;
applying to the subject a nicotine patch;
determining if the subject has a reduction in the one or more symptoms, and
if the subject has a reduction of the one or more symptoms, then providing the subject with a composition of claim 1.

24. The method of claim 23, wherein the symptoms are determined by at least one of: a bowel movement journal reflects constipation (fewer than one bowel movement per day); Small bowel manometry reflects low motility; Imaging with fluoroscopy reveals ileocecal valve dysfunction (ileus); Heidelberg testing reveals low stomach acid production; HIDA scan reveals low gallbladder ejection fraction; stool quality or color, steatorrhea—oil at the top of the toilet water after a bowel movement, reveals pancreatic enzyme deficiency; or Tachycardia at rest;
the nicotine patch stimulates post-ganglionic nicotinic acetylcholine receptors or the nicotinic acetylcholine receptors on the organs that results in correction of at least one of: constipation, gastroparesis, idiopathic gastrointestinal dysmotility, ileocecal valve dysfunction (“ileus”), low stomach acid production, low gallbladder ejection fractions, pancreatic enzyme deficiency, or tachycardia; or
the nicotine patch is placed on an abdomen or trunk.

25. The method of claim 23, further comprising checking the effect of the abnormal testing for normalization after 30 minutes, 2, 3, 4, 5, 6, 7, 8, 9, 10, 11, 12, 13, 14, 15, 16, 17, 18, 19, 20, 21, 22, 23, or 24 hours.

26. The method of claim 23, wherein a response to the nicotine patch is indicative that a post-ganglionic receptor is viable, wherein a subject is provided a composition according to claim 1.
